(19) United States
(12) Patent Application Publication
(10) Pub. No.: US 2010/0236929 A1
(43) Pub. Date: Sep. 23, 2010

(54) Droplet Actuators, Systems and Methods

(75) Inventors: Michael G. Pollack, Durham, NC (US); Vamsee K. Pamula, Durham, NC (US); Vijay Srinivasan, Durham, NC (US); Krishnendu Chakrabarty, Chapel Hill, NC (US); Tao Xu, Cary, NC (US)

Correspondence Address: ADVANCED LIQUID LOGIC, INC.;C/O WARD AND SMITH, P.A., 1001 COLLEGE COURT, P.O. BOX 867, NEW BERN, NC, 28563-0867, US

(73) Assignees: ADVANCED LIQUID LOGIC, INC., Research Triangle Park, NC (US); DUKE UNIVERSITY, Durham, NC (US)

(21) Appl. No.: 12/681,862

(22) PCT Filed: Oct. 16, 2008
(86) PCT No.: PCT/US2008/080216

§ 371 Date: Apr. 19, 2010

Related U.S. Application Data
(60) Provisional application No. 60/980,839, filed on Oct. 18, 2007.
(60) Provisional application No. 60/980,841, filed on Oct. 18, 2007.
(60) Provisional application No. 60/980,844, filed on Oct. 18, 2007.
(60) Provisional application No. 61/016,477, filed on Dec. 23, 2007.
(60) Provisional application No. 61/016,479, filed on Dec. 23, 2007.

(51) Int. Cl. G01N 27/26; G01N 33/53

(52) U.S. Cl. 204/450; 204/600

(57) ABSTRACT

A droplet actuator with arrays of electrodes electrically coupled to a number of controllable voltage sources that is less than the number of electrodes. A method of defining partitions for pin layouts in a droplet actuator for a specific assay, the method including: defining droplet traces for the assay; and defining a guard ring along the traces. Other methods, systems, droplet actuators, and algorithms are also provided.

### RELATED PATENT APPLICATIONS

This application claims priority to U.S. Patent Application No. 60/980,839, entitled “Droplet trace-based array partitioning and pin assignment algorithm,” filed on Oct. 18, 2007; U.S. Patent Application No. 60/980,841, entitled “Broadcast electrode-addressing for pin-constrained multi-functional digital microfluidic biochips,” filed on Oct. 18, 2007; U.S. Patent Application No. 61/016,479, entitled “Broadcast electrode-addressing for pin-constrained multi-functional digital microfluidic biochips,” filed on Dec. 23, 2007; U.S. Patent Application No. 60/980,844, entitled “A cross-referencing based droplet manipulation method for high throughput and pin-constrained digital microfluidic arrays,” filed on Oct. 18, 2007; and U.S. Patent Application No. 61/016,477, entitled “A cross-referencing based droplet manipulation method for high throughput and pin-constrained digital microfluidic arrays,” filed on Dec. 23, 2007; the entire disclosures of which are incorporated herein by reference.

### GOVERNMENT INTEREST

This invention was made with government support under IIS-0312352 and CCF-0541055 awarded by the National Science Foundation and CA114993-01 awarded by the National Institutes of Health. The United States Government has certain rights in the invention.

### FIELD OF THE INVENTION

The invention relates to algorithms for use in configuring droplet actuator layouts and routing droplets on droplet actuators. The invention relates to a partitioning algorithm for use in configuring droplet actuator layouts in which the number of droplet operation electrodes is less than the number of switches controlling such electrodes. The invention also relates to a broadcast-addressing-based design technique for pin-constrained multi-functional droplet actuators. The invention further relates to a droplet operations method that is based on a “cross-referencing” addressing method that uses “row” and “columns” to access electrodes.

### BACKGROUND

Pin-constrained design of droplet actuators was recently proposed and analyzed in [1]. The number of control pins is minimized by using a multi-phase bus for the fluidic pathways. Every nth electrode in an n-phase bus is electrically connected. Thus, only n control pins are needed for a transport bus, irrespective of the number of electrodes that it contains. Although the multi-phase bus method is useful for reducing the number of control pins, it is only applicable to a one-dimensional (linear) array.

An alternative method based on a cross-reference driving scheme is presented in [2]. This method is reported to allow control of an N×M grid array with only N+M control pins. The electrode rows are patterned on both the top and bottom plates, and placed orthogonally. In order to drive a droplet along the X-direction, electrode rows on the bottom plate serve as driving electrodes, while electrode rows on the top serve as reference ground electrodes. The roles are reversed for movement along the Y-direction. This cross-reference method facilitates the reduction of control pins. However, it requires a special electrode structure (i.e., both top and bottom plates containing electrode rows), which results in increased manufacturing cost for disposable microfluidic droplet actuators. Moreover, this design is not suitable for high-throughput assays because droplet movement is inherently slow.

More recently, a promising design method based on array partitioning has been proposed for pin-constrained droplet actuators [3]. The electrode array is divided into several partitions and sets of pins are determined, where each set of pins correspond to a partition and all the sets are of the same size. For example, if a droplet actuator of arbitrary size is divided into six partitions and five pins are allocated per set, only 5×6=30 pins are needed to independently address the individual unit cells of the array. By carefully controlling the number of partitions, the total number of pins is reduced significantly compared to the direct-addressing scheme.

However, the design method presented in [3] suffers from several drawbacks. First, the array partitioning in [3] is ad-hoc and no systematic algorithm has thus far been presented. Secondly, microfluidic modules such as mixers, splitters, and detectors are not considered in the ad-hoc partitioning method; improved designs are required to facilitate handling these modules separately. Moreover, the partitioning method assumes a priori that partitions do not overlap; this restriction can be a limitation for many bioassays. Finally, no pin-assignment algorithm is presented in [3].

FIG. 1 illustrates the problem of electrode interference. This problem can appear in arrays in which multiple electrodes are controlled using a single pin. For example, assume that a droplet rests on an electrode (unit cell) and two of its neighbors are connected to the same pin. Recall that to move the droplet to one of two neighbors (i and j) that share the same pin, we must deactivate the electrode where the droplet rests and activate the destination electrode i. However, when electrode i is activated, the other neighbor electrode j is also activated since it shares the same pin with electrode i. In this case, the droplet undergoes a split, instead of being moved to electrode i. This problem can be solved by addressing each electrode and its neighbors with distinct pins. Since one electrode can have at most four neighbors in a two-dimensional array, the minimum number needed is five. Recent experimental studies have shown that five independent pins are adequate to route a droplet to any place on the droplet actuator for single droplet transport operation [3].

When multiple droplet operations are performed simultaneously on the droplet actuator, a pin-constrained layout may also result in unintentional droplet movement or other unintended consequences. For the example in FIG. 2, electrode interference will occur if an attempt is made to move Droplet Dᵢ and let Droplet Dⱼ stay where it is. To move Dᵢ one cell downwards, we need to activate Pin 8 and deactivate Pin 1. To hold Droplet Dⱼ, we need to activate Pin 3. However, since both Pin 3 and Pin 8 are charged, Dⱼ will be split unintentionally. This type of problem is referred to as electrode interference.

Electrode interference can be solved by “virtually” partitioning the array into regions, with each of them having only one activated cell at any point in time. Mutually-exclusive sets of pins are utilized for conducting droplet operations in different regions. The partitions can be viewed as subarrays that can contain at most one droplet. Regardless of size, a two-dimensional array only needs five independent pins to ensure full control of a single droplet. By using different sets of five pins for electrode control in different partitions, electrode interference among partitions can be avoided. Therefore, for the partitioned array, the number of droplets that can be simultaneously transported without stall cycles is equal to the number of partitions, and the total number of control pins needed is equal to five times the number of partitions. The above partitioning solution was proposed recently in [3].

However, both array partitioning and the assignment of control pins to electrodes in [3] are done in an ad-hoc manner. No systematic algorithms have been proposed thus far to implement the partitioning-based pin-assignment method and incorporate it in automated design tools.

The emergence of microfluidic droplet actuators has led to the automation of laboratory procedures in biochemistry and the miniaturization of laboratory instruments [4,5]. Compared to traditional bench-top procedures, microfluidic droplet actuators offer the advantages of low sample and reagent consumption, less likelihood of error due to minimal human intervention, high throughput, and high sensitivity. These lab-on-a-droplet actuator devices are now being advocated for a wide range of applications such as high-throughput DNA sequencing, immunoassays and clinical chemistry, environmental toxicity monitoring and the detection of airborne chemicals, detection of explosives such as TNT, and point-of-care diagnosis of diseases [6,7].

Demonstrated applications of digital microfluidics include the on-droplet actuator detection of explosives such as commercial-grade 2,4,6-trinitrotoluene (TNT) and pure 2,4-dinitrotoluene [8], automated on-droplet actuator measurement of airborne particulate matter [9,10], and colorimetric assays [1]. Digital microfluidic droplet actuators are being designed for on-droplet actuator gene sequencing through synthesis [10], protein crystallization, clinical diagnostics for high throughput with low sample volumes, and integrated hematology, pathology, molecular diagnostics, cytology, microbiology, and serology onto the same platform [11].

Currently, most commercially-available droplet actuators rely on either continuous fluid flow in etched microchannels or microarrays [5, 12]. Fluid flow is controlled either using micropumps and microvalves [5] or using electrokinetics [13]. An alternative category of microfluidic droplet actuators relies on the principle of electrowetting-on-dielectric. Discrete droplets of nanoliter volumes can be manipulated in a “digital” manner on a two-dimensional electrode array. Hence this technology is commonly referred to as “digital microfluidics” [4].

A typical digital microfluidic droplet actuator commonly consists of a two-dimensional electrode array [4]. A unit cell in the array includes a pair of electrodes that acts as two parallel plates. The bottom plate contains a patterned array of electrodes, and the top plate is coated with a continuous ground electrode. A droplet rests on a hydrophobic surface over an electrode, as shown in FIG. 2. It is moved by applying a control voltage to an electrode adjacent to the droplet and, at the same time, deactivating the electrode just under the droplet. This electronic method of wettability control creates interfacial tension gradients that move the droplets to the charged electrode. Using the electrowetting phenomenon, droplets can be moved to any location on a two-dimensional array.

By varying the patterns of control-voltage activation, many fluid-handling operations such as droplet dispensing, merging, splitting, mixing, localized heating, and incubation can be executed on-droplet actuator in a programmable fashion. For example, mixing can be performed by routing two droplets to the same location and then turning them about some pivot points [14]. The digital microfluidic platform offers the additional advantage of flexibility, referred to as reconfigurability, since fluidic operations can be performed anywhere on the array. Droplet routes and operation scheduling result are programmed into a microcontroller that drives electrodes in the array. In addition to electrodes, optical detectors such as LEDs and photodiodes are also integrated in digital microfluidic arrays to monitor colorimetric bioassays [15].

Electrodes are typically connected to control pins for electrical actuation. A number of prototype droplet actuators use a direct-addressing scheme for the control of electrodes [8, 16]. Each electrode is connected to a dedicated control pin; it can therefore be activated independently. This method allows the maximum freedom of performing droplet operations, but it necessitates an excessive number of control pins for practical droplet actuators. As more bioassays are concurrently executed on digital microfluidic platforms [1, 17], system complexity and the number of electrodes is expected to increase steadily. Recently, a droplet-based droplet actuator that embeds more than 600,000 20 μm by 20 μm electrodes, and uses dielectrophoresis for droplet operations and control, has been demonstrated [18]. The large number of control pins and the associated interconnect-routing problem significantly adds to product cost.

To address the need for low-cost, PCB technology has been proposed to inexpensively mass-fabricate digital microfluidic droplet actuators [19]. This inexpensive manufacturing technique allows for the building of disposable PCB-based microfluidic droplet actuators that can be easily plugged into a controller circuit board that can be programmed and powered via a standard USB port. However, a large number of independent control pins necessitates multiple PCB layers, which adds significantly to the product cost. Thus, the design of pin-constrained digital microfluidic arrays is of great practical importance for the emerging marketplace. Of particular interest are design techniques that provide high throughput despite the availability of a limited number of control pins.

Electrode-addressing methods that allow the control of microfluidic arrays with a small number of pins are now receiving attention. The method presented in [3, 20] uses array partitioning and careful pin-assignment to reduce the number of control pins. However, this method is specific to a target biofluidic application. An alternative design uses row- and column-addressing, a technique referred to as “cross referencing”. An electrode is connected to two pins, corresponding to a row and a column, respectively [2].

Research on design-automation techniques for microfluidic droplet actuators has gained momentum in recent years, in part due to the enthusiasm generated from advances in digital microfluidic technology. In [16], classical architectural- and geometric-level synthesis methods are adapted for the automated design of droplet actuators. A unified synthesis method, which combines scheduling, resource binding, and module placement, has been proposed in [16]. Systematic droplet routing strategies have also been developed [21, 22]. These early design automation techniques are useful for droplet actuator design and rapid prototyping, but they all rely on the availability of a direct-addressing scheme [23, 24]. However, as discussed hereinabove, direct-addressing suffers from the drawback of higher wiring complexity.

Pin-constrained design for digital microfluidics was addressed in [20]. This method uses array partitioning and careful pin-assignment to reduce the number of control pins. However, it requires detailed information about the scheduling of assay operations, microfluidic module placement, and droplet routing pathways. Thus, the array design in such cases is specific to a target biofluidic application.

In another method proposed in [1], the number of control pins for a fabricated electrowetting-based droplet actuator is minimized by using a multi-phase bus for the fluidic pathways. Every nth electrode in an n-phase bus is electrically connected, where n is small number (typically n=4). Thus, only n control pins are needed for a transport bus, irrespective of the number of electrodes that it contains. Although the multi-phase bus method is useful for reducing the number of control pins, it is only applicable to a one-dimensional (linear) array.

An alternative method based on a cross-reference driving scheme is presented in [2]. This method allows control of an N×M grid array with only N+M control pins. The electrode rows are patterned on both the top and bottom plates, and placed orthogonally. An electrode is activated by highlighting the column and row pins it resides on. However, due to electrode interference, this design cannot handle the simultaneous movement of more than two droplets. The resulting serialization of droplet movement is a serious drawback for high-throughput applications such as DNA sequencing, air-quality monitoring, multiplexed immunoassays, and proteomic analysis [25]. Higher throughput can be achieved for such arrays using a graph-theoretic optimization technique [26]. However, this design requires a special electrode structure (i.e., both top and bottom plates contain electrode rows), which results in increased manufacturing cost.

In digital microfluidic droplet actuators, droplets of nanoliter volumes, which contain biological samples, are typically manipulated on a two-dimensional electrode array [4]. A unit cell in the array includes a pair of electrodes that acts as two parallel plates. In most prototype digital microfluidic droplet actuators based on the ct-addressing scheme, the bottom plate contains a patterned array of individually controlled electrodes, and the top plate is coated with a continuous ground electrode. A droplet rests on a hydrophobic surface over an electrode. Recently, coplanar microfluidic devices, i.e., arrays without a top plate, have also been demonstrated [27]. Using the electrowetting phenomenon, droplets can be moved to any location on a two-dimensional array. An alternative category of digital microfluidic droplet actuators utilizes orthogonally-placed pin rows on top and bottom plates. A unit cell can be activated by selecting orthogonally positioned pins on the top and bottom plates which cross at this cell.

An alternative method based on a cross-reference driving scheme is presented in [2]. In order to drive a droplet along the X-direction, electrode rows on the bottom plate serve as driving electrodes, while electrode rows on the top serve as reference ground electrodes. The roles are reversed for movement along the Y-direction, as shown in FIG. 3. This cross-reference method facilitates the reduction of control pins. However, due to electrode interference, this design cannot handle the simultaneous movement of more than two droplets. The resulting serialization of droplet movement is a serious drawback for high-throughput applications.

The minimization of the assay completion time, i.e., the maximization of throughput, is essential for environmental monitoring applications where sensors can provide early warning. Real-time response is also necessary for surgery and neo-natal clinical diagnostics. Finally, biological samples are sensitive to the environment and to temperature variations, and it is difficult to maintain an optimal clinical or laboratory environment on droplet actuator. To ensure the integrity of assay results, it is therefore desirable to minimize the time that samples spend on-droplet actuator before assay results are obtained. Increased throughout also improves operational reliability. Long assay durations imply that high actuation voltages need to be maintained on some electrodes, which accelerate insulator degradation and dielectric breakdown, reducing the number of assays that can be performed on a droplet actuator during its lifetime.

### SUMMARY OF THE INVENTION

The invention provides a droplet actuator, systems including such droplet actuators, and methods of making and using such droplet actuators. The invention provides a droplet actuator including a configuration of droplet operations electrodes, wherein the droplet operations electrodes are grouped into electrode subsets in which electrodes in each electrode subset are electrically coupled together. The invention also provides methods of conducting droplet operations using such droplet actuators.

The invention also provides a droplet actuator and methods for proforming droplet operations using an electrical field generated by a droplet actuator having a plurality of control pins used to electrically actuate a plurality of electrodes configured to communicate the electrical filed to the droplet, the method comprising: receiving a plurality of respective activation sequences for each electrode of a plurality of electrodes; automatically identifying based on the received activation sequences a compatible activation sequence that is compatible with the plurality of respective activation sequences; communicating the compatible activation sequence to the plurality of electrodes using a common control pin of a plurality of control pins; and proforming droplet operations according to the compatible activation sequence. In an alternative embodiment of the invention, the method includes identifying a compatible activation sequence that is compatible with the plurality of respective activation sequences by logically manipulating a signal value used to determine a status of an electrode at a given time-step of the respective activation sequence of the electrode. The droplet actuator of the invention includes a processor in communication with both the plurality of electrodes and control pins and configured to receive a plurality of respective activation sequences for each electrode of a plurality electrodes, to automatically identify based on the received activation sequences a compatible activation sequence that is compatible with the plurality of respective activation sequences, to communicate the compatible activation sequence to the plurality of electrodes using a common control pin of a plurality of control pins, and to proform droplet operations according to the compatible activation sequence.

The invention further provides a droplet actuator and methods of proforming droplet operations using an electrical field generated by a droplet actuator having rows and columns corresponding to electrodes configured to communicate the electrical field the droplet, the method comprising: logically grouping a plurality of droplets into first and second groups; and concurrently moving a plurality of droplets of the first group, while a droplet of the second group is stationary. The droplet actuator of the invention includes a processor in communication with the plurality of electrodes and configured to logically group a plurality of droplets into first and second groups, and to concurrently initiate movement of a plurality of droplets in the first group, while a droplet of a second group is stationary.

Other aspects of the invention will be apparent from the ensuing definitions and detailed description of the invention.

### DEFINITIONS

As used herein, the following terms have the meanings indicated.

“Activate” with reference to one or more electrodes means effecting a change in the electrical state of the one or more electrodes which, in the presence of a droplet, results in a droplet operation.

“Droplet” means a volume of liquid on a droplet actuator that is at least partially bounded by filler fluid. For example, a droplet may be completely surrounded by filler fluid or may be bounded by filler fluid and one or more surfaces of the droplet actuator. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator.

“Droplet Actuator” means a device for manipulating droplets. For examples of droplet actuators, see U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on filed on Jan. 30, 2006; U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000, both to Shenderov et al.; Pollack et al., International Patent Application No. PCT/US2006/047486, entitled “Droplet-Based Biochemistry,” filed on Dec. 11, 2006, the disclosures of which are incorporated herein by reference. Methods of the invention may be executed using droplet actuator systems, e.g., as described in International Patent Application No. PCT/US2007/009379, entitled “Droplet manipulation systems,” filed on May 9, 2007. In various embodiments, the manipulation of droplets by a droplet actuator may be electrode mediated, e.g., electrowetting mediated or dielectrophoresis mediated.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; condensing a droplet from a vapor; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to size of the resulting droplets (i.e., the size of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. In various embodiments, the droplet operations may be electrode mediated, e.g., electrowetting mediated or dielectrophoresis mediated.

“Filler fluid” means a fluid associated with a droplet operations substrate of a droplet actuator, which fluid is sufficiently immiscible with a droplet phase to render the droplet phase subject to electrode-mediated droplet operations. The filler fluid may, for example, be a low-viscosity oil, such as silicone oil. Other examples of filler fluids are provided in International Patent Application No. PCT/US2006/047486, entitled, “Droplet-Based Biochemistry,” filed on Dec. 11, 2006; and in International Patent Application No. PCT/US2008/072604, entitled “Use of additives for enhancing droplet actuation,” filed on Aug. 8, 2008.

“Washing” with respect to washing a magnetically responsive bead means reducing the amount and/or concentration of one or more substances in contact with the magnetically responsive bead or exposed to the magnetically responsive bead from a droplet in contact with the magnetically responsive bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Other embodiments are described elsewhere herein, and still others will be immediately apparent in view of the present disclosure.

The terms “top” and “bottom” are used throughout the description with reference to the top and bottom substrates of the droplet actuator for convenience only, since the droplet actuator is functional regardless of its position in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### BRIEF DESCRIPTION OF THE DRAWINGS

FIG. 1 illustrates the problem of electrode interference;

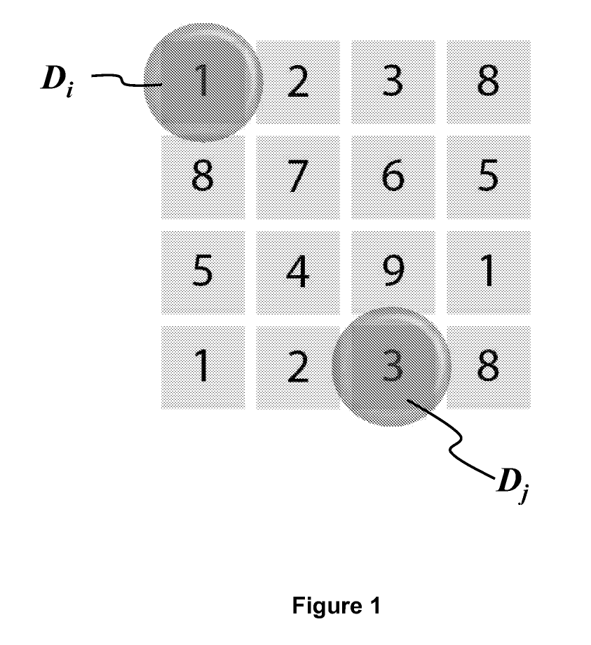

FIGS. 2A and 2B illustrate a schematic view of a 2-D digital microfluidic array and a side view of a unit cell of a droplet actuator, respectively;

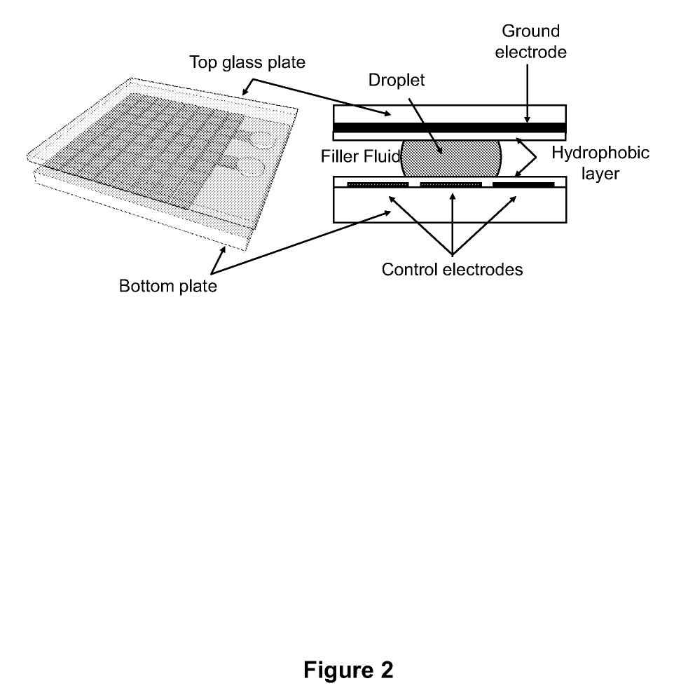

FIG. 3 illustrates cross sections of a cross-referencing microfluidic device that uses single-layer driving electrodes on both top and bottom plates;

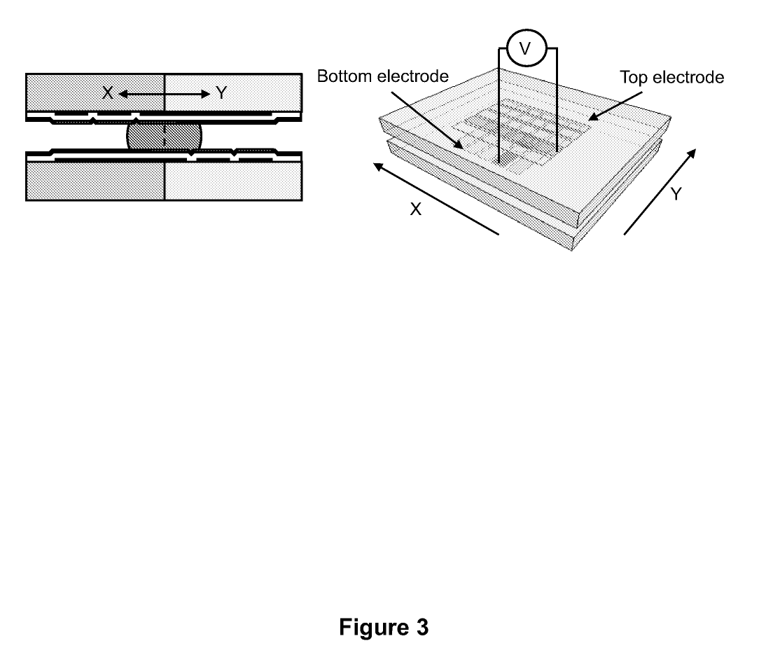

FIGS. 4A and 4B illustrate a trace extraction example involving droplet operations with two separate droplets on the electrode array;

FIGS. 5A and 5B illustrate direct-addressing in overlapping partitions;

FIGS. 6A and 6B illustrate a pin assignment example for a mixer and a splitter, respectively;

FIG. 7 illustrates mapping an array to an undirected graph;

FIG. 8 illustrates a Bagua or a tiled square;

FIG. 9 illustrates the derivation of a Bagua repetition;

FIG. 10 illustrates pin assignment to cells in a Bagua repetition;

FIG. 11 illustrates the wiring layout provided by the Connect-5 algorithm;

FIG. 12 illustrates a digital microfluidic droplet actuator for a multiplexed biochemical assay that contains a 15×15 electrode array;

FIG. 13 illustrates the partition assignments for the droplet actuator of FIG. 12;

FIG. 14 illustrates the pin assignments for the droplet actuator of FIG. 12;

FIG. 15 illustrates an example of a “don't-care” in electrode activation;

FIGS. 16A and 16B illustrate an example of activation sequence calculation (a) routing and layout information (b) calculated activation;

FIG. 17 illustrates mapping of activation sequences to an undirected graph;

FIG. 18 illustrates a sequencing graph model for a multiplexed bioassay. S₁, S₂ are samples, R₁, R₂ are reagents, M₁˜M₄ are mixing operations, and D₁˜D₄ are detection operations;

FIG. 19 illustrates mapping of a multiplexed bioassay to a 15×15 array;

FIG. 20 illustrates a schedule result for the multiplexed bioassay;

FIG. 21 illustrates broadcast addressing for the multiplexed assay droplet actuator;

FIG. 22 shows a comparison of assay completion times;

FIG. 23 illustrates a sequencing graph for the mixing stage of PCR;

FIG. 24 illustrates mapping of the PCR assay on a 15×15 array;

FIG. 25 illustrates a schedule for the PCR assay;

FIG. 26 illustrates broadcast addressing for the PCR droplet actuator;

FIG. 27 illustrates a sequencing graph for the protein assay;

FIG. 28 illustrates a protein dilution droplet actuator layout;

FIG. 29 illustrates a schedule for the protein dilution assay, Dlt—dilution, Mix—mixing, Opt—optical detection;

FIG. 30 illustrates broadcast addressing for the protein-dilution droplet actuator;

FIG. 31 illustrates a multi-functional droplet actuator layout;

FIG. 32 illustrates the problem of electrode interference. H/L stands for high/low voltage pairs to activate the cells, and unselected row/column pins are left floating (F);

FIG. 33 illustrates an example of electrode interference within the same row;

FIG. 34 illustrates an example of destination-cell-based categorization;

FIG. 35 illustrates an example of the concurrent movement of a group of droplets;

FIG. 36 illustrates an example of electrode interference due to asynchronous processing of proforming multiple droplet operations;

FIG. 37 illustrates mapping destination cell layout to undirected graph;

FIG. 38 illustrates a 15×15 array used for multiplexed bioassays;

FIG. 39 illustrates a droplet operations step in direct-addressing routing; and

FIGS. 40A and 40B illustrate implementing the step of FIG. 39 by two substeps using the proposed cross-referencing based method: (a) Substep 1; (b) Substep 2.

### DESCRIPTION

The invention provides algorithms for manipulation of droplets on a droplet actuator. The invention also provides droplet actuators configured for execution of droplet algorithms. The invention also provides methods of manipulating droplets according to such algorithms. Further, the invention provides systems programmed to execute methods according to such algorithms, and electronic storage media comprising such software. Other aspects of the invention will be apparent from the ensuing discussion.

8.1 Droplet-Trace-Based Array Partitioning and a Pin Assignment Algorithm

The invention provides an algorithm based on the concept of droplet trace, which unifies array partitioning and pin assignment. Partitioning can effectively avoid electrode interference if each partition includes only one droplet. The partitioning criterion provided here ensure that at most one droplet is included in each partition. Partitions with no droplets (at any point in time) should generally also be avoided because this scenario results in unnecessary expense of including pins that are not used, i.e., no droplet operation involving activation of an electrode is conducted in the region with the additional set of pins assigned to it. Hence it is preferable that each partition has exactly one droplet in it.

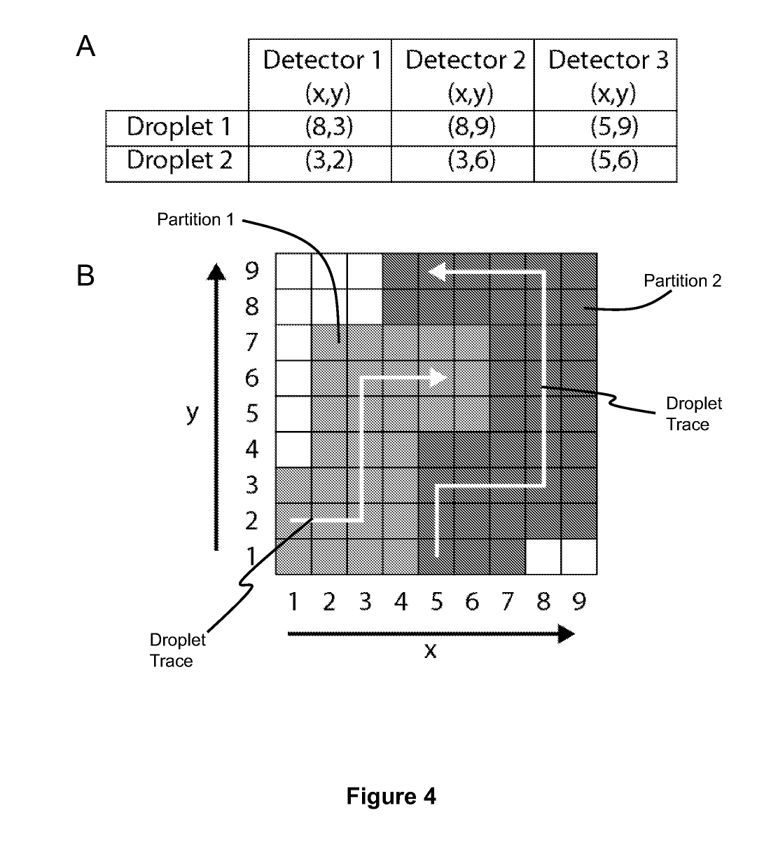

Based on these preferred conditions, the invention provides an approach in which the droplet trace, defined as the set of cells traversed by a single droplet, is used for generating the array partitions. Pin assignment can be viewed as the last step in system synthesis, information about module placement and droplet routing is available a priori. The droplet trace can be obtained from the droplet routing information and the placement of the modules to which each droplet is routed. FIGS. 4A and 4B illustrate a trace extraction example involving droplet operations with two separate droplets on the electrode array. Both of these are required to be detected by an optical sensor three times in a specific bioassay. The placement of these detectors is shown in FIG. 4A. The droplet traces, i.e., the path taken by droplets, are illustrated by the arrows in FIG. 4B. For each droplet, a partition is created composed of all the cells on its trace as well as the cells adjacent to the trace. The adjacent cells are included to form a “guard ring” along the trace to avoid inadvertent mixing and movement. The guard rings are a consequence of the fluidic constraint described in [28].

Note that in FIG. 4B, there are two “white” regions that belong to neither partition. They are referred to as “don't-care” regions because they are similar to the “don't-care” terms in logic synthesis; they can either be assigned to any partition or they can together form an additional partition if multi-droplet-operation modules, e.g. mixers, can be positioned in them.

In order to reduce the number of partitions, a time-division pin-sharing method is provided. According to this aspect of the invention, partitions are merged that have no overlapping time spans. “Time span” for a partition means the period of time during which it contains a droplet. The time spans for all the partitions can be easily calculated from the operation schedule, module placement and droplet routing results [28]; the overlaps can then be readily determined Partitions with non-overlapping time spans are merged to form a larger partition. This check-merge procedure continues until all partition pairs overlap in their time spans. By reducing the number of partitions, the approach of the invention reduces the number of control pins needed for the array. Note that droplet traces may have spatial overlap, i.e., they may intersect at one or more unit cells on the array. In this case, the requirement of one droplet per partition is not met and electrode interference may occur. This problem is handled by simply modifying the partitioning result.

In some embodiments, droplet traces may intersect on the array. In other words, partitions derived by the proposed method overlap in some regions. Sets of pins from an “overlapping” partition cannot be used in the overlapped region since the reuse of the pins leads to electrode interference. One solution to this problem is to make the overlapping region a new partition, referred to as the overlapping partition, and use direct-addressing for it. Again, time-division pin-sharing (TDPS) can be used to reduce the number of pins since pin sets of the other (non-overlapping) partitions can be candidates for direct-addressing in the overlapping partition.

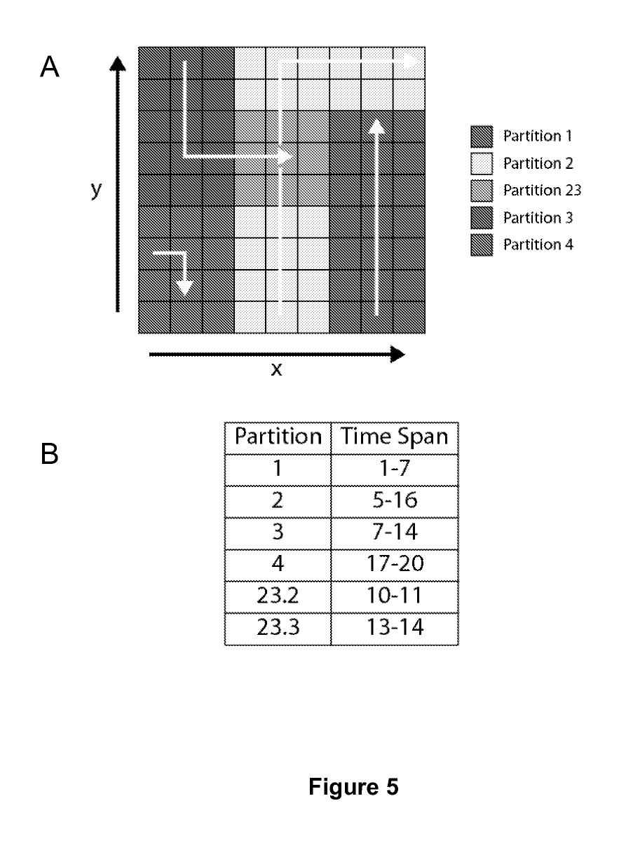

An example of this approach is shown in FIGS. 5A and 5B. The droplet traces are first derived from the droplet routing information. Partitions 1, 2, 3, and 4 are assigned accordingly. Partition 2 and Partition 3 overlap with each other as shown. Thus a new Partition 23 is created. From the scheduling result in FIG. 5B, the time span for Partition 23 is found to be 10-14 s. Next the time spans for Partitions 1 and 4 are checked and it is seen that their time spans do not overlap with that for Partition 23. Hence the two sets of pins (a total of 2×5=10 pins) in Partitions 1 and 4 can be used to directly address the nine electrodes in Partition 23.

Partitions that share pins with the overlapping partition are empty while droplet operations occur in the overlapping partition. Therefore, the sharing of pins in these cases does not lead to electrode interference. By introducing the concept of TDPS, the invention enables a significant reduction in the number of pins required for independent addressing. The concept of TDPS can also be applied in the spatial dimension to the operations inside the overlapping region to further reduce the number of control pins.

Once a spatially overlapping region is found, it is possible to determine if there are temporally overlapping droplets in this region. Depending on the outcome of this procedure, a spatial overlap region can be then divided into two groups—a spatially overlapping but temporally non-overlapping (SOTN) region, and a spatially overlapping as well as temporal overlapping (SOTO) region. For SOTO regions, direct-addressing is used. For SOTN regions, even though droplets traces cross each other, different droplets are sequenced in time (one after the other), i.e., at any point in time, there is at most one droplet inside the region. In this case, a pin set with the minimum size (k=5) for single droplet operation is assigned to this SOTN region.

Again, referring to the above example of FIGS. 5A and 5B for illustration, Table 1 shows the schedule information needed for carrying out the temporal check for the overlapping region:

TABLE 1

| Partition | Time Span |
|---|---|
| 1 | 1-7 |
| 2 | 5-12 |
| 3 | 7-23 |
| 4 | 17-20 |
| 23 | 10-14 |

Partitions 23.2 and 23.3 represent droplet operations involving Droplet 2 and Droplet 3 in Partition 23 respectively. Table 1 shows that the time spans for these partitions do not overlap, thus five pins (in contrast to the nine pins needed for direct-addressing) are adequate for the overlapping partition.

### 8.1.1 Extended Partitioning Algorithm

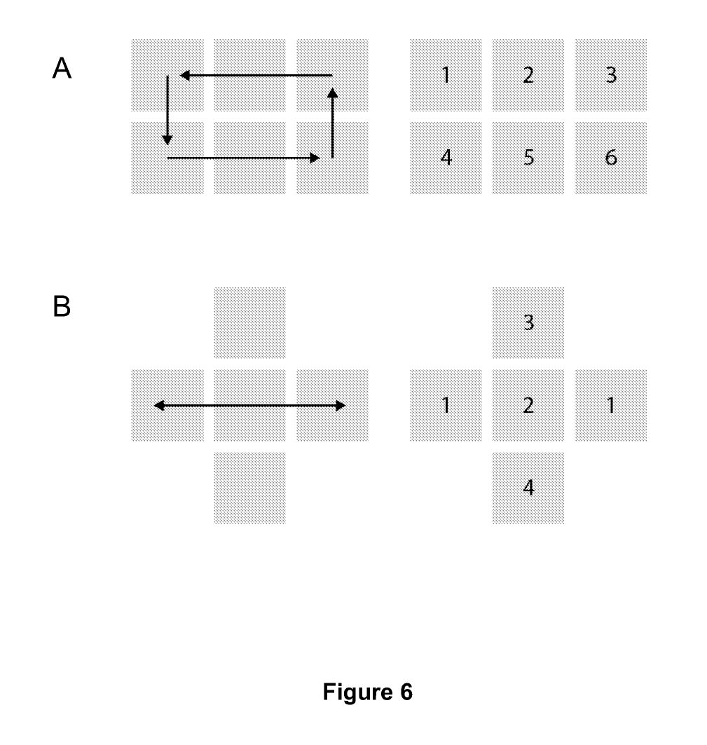

The invention also provides an extension of the partitioning algorithm that does not require module placement information. The method described thus far requires knowledge of the placement information for modules that handle multiple droplets, such as mixers and splitters to determine the droplet traces. This aspect requires only on the schedule of operations and droplet routing results to indirectly determine module placement. For example, the mixing operation can be viewed as two droplets being routed together along an identical path simultaneously with the start point in the mixer region. Similarly, droplet splitting can be viewed as two droplets sharing the same start point in both the time and space domains. Therefore, mixer regions can be identified by checking whether droplet traces exactly overlap instead of just intersecting each other in the same time span; a splitter can be recognized in a similar manner. As a result, overlapping partitions can be assigned to mixers and splitters. Note that splitting and mixing can both be viewed as deliberate (desired) electrode interference. Thus, though multiple droplet operations occur in mixer or splitter regions, five control pins are sufficient, as shown in FIG. 6, which illustrates a pin assignment example for a mixer (FIG. 6A) and a splitter (FIG. 6B). In this way, the number of pins can be further reduced.

### 8.1.2 Pin Assignment Using the Connect-5 Algorithm

The method described thus far provides a partitioning method for droplet actuator electrode arrays. Each partition is assigned a pin set. The invention also provides a solution to the problem of how to map control pins to the electrodes in a partition. The algorithm is based on a strategy of the Connect-5 (Gomoku) board game [29], thus it is referred to as the Connect-5 algorithm.

The sets of pins assigned to the partitions belong to two groups according to their cardinality, i.e., the minimum for single droplet operation (k=5) or the number of pins required for direct-addressing. For the first case, it is possible to focus on the pin assignment problem, since pin assignment for direct-addressing is straightforward (there exists a simple one-to-one mapping between pins and electrodes).

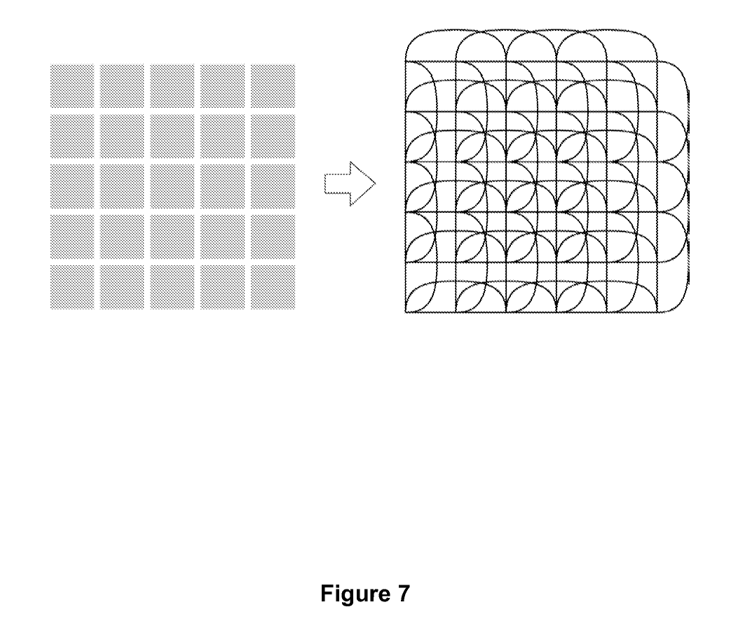

The goal of this approach is to ensure that any five adjacent unit cells (a central cell and its four neighbors) that form a “cross” are assigned distinct pins. We refer to the above constraint as the “cross constraint”. The pin assignment problem under cross constraints can be mapped to the famous vertex coloring problem in graph theory [30]. The problem here is to obtain a 5-coloring of the graph derived from a partition, as shown in FIG. 7, which illustrates mapping an array to an undirected graph. The unit cells in the partition are mapped to vertices and any two cells that belong to a “cross” are connected by an edge. The graph corresponding to a partition is referred to as the partition graph.

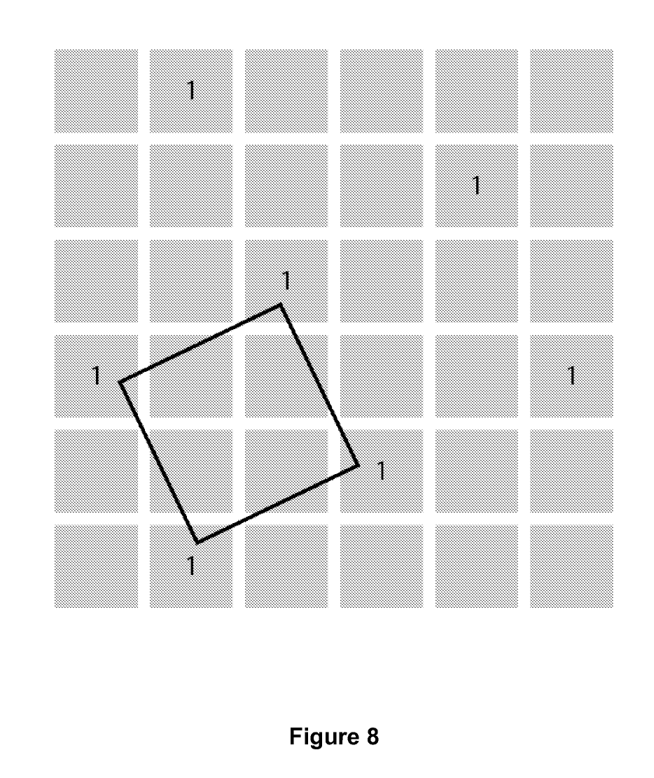

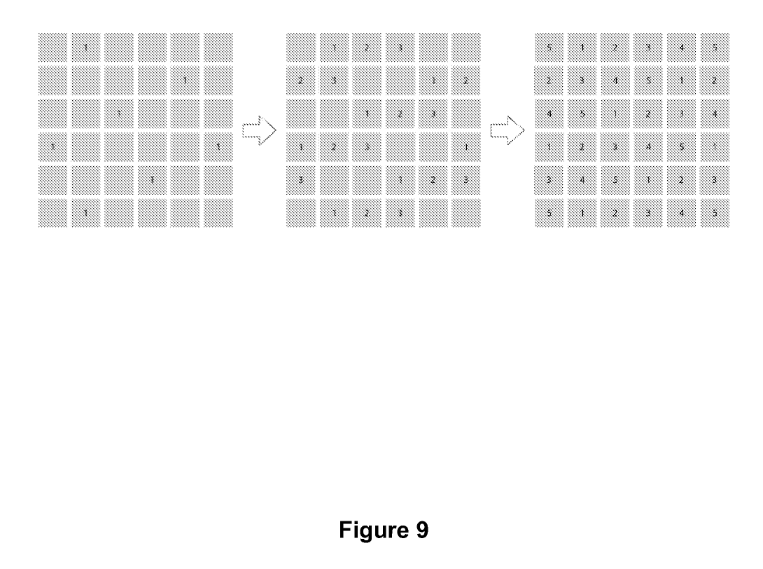

The graph coloring problem, which involves the determination of the chromatic number χ(G) for a graph G, is known to be NP-complete [30]. However, if χ(G) or the number of colors to be used is known, as in the case here, there exists efficient algorithm for graph coloring. However, the regular structure of the partitions can be used to solve the problem more efficiently using tiling. This approach allows us to use a regular distribution of pins, a layout feature that is not directly obtained from graph coloring. The tile (or template) used here is referred to as “Bagua,” a Chinese game strategy for the Connect-5 board game [29]. A Bagua is a tiled square, as shown in FIG. 8. By repeating placing Bagua structures next to each other until the partition boundaries are reached, a Bagua repetition is derived as shown in FIG. 9. The tiling using Bagua repetitions forms the basis for the Connect-5 algorithm.

Five copies of Bagua repetitions are sufficient to cover a partition of any size. This is because of the following property of a Bagua repetition: vertices connected to the same (shared) pin appear after exactly five cells in the same row or column of the partition. The partition can be covered with Bagua repetitions by simply taking a Bagua repetition and shifting it one cell along an arbitrary direction, e.g., upwards, then assigning it to another control pin and repeating this step four times, as shown in FIG. 9. Note that, although the shifting direction is arbitrarily selected at the start of the tiling process, once chosen it must be consistent over the shifting steps.

As shown in FIG. 9, the pin assignment that results from the shifting of Bagua repetition satisfies a cyclic property, i.e., each row is a cyclic repetition of an ordered sequence, and it is also a shifted copy (shift by two cells) of the previous row. This cyclic property provides an easy way to implement the Connect-5 algorithm.

To start, the first row of a partition is selected. Pins are assigned in a fixed cyclic order until the boundary of the partition is reached. Then in the next row, the same order is used for but with a 2-cell-shift to the left/right. The procedure continues until all cells in the partition have been assigned pins. Recall that the shifting direction, once chosen, must remain fixed during the assignment procedure for a given partition.

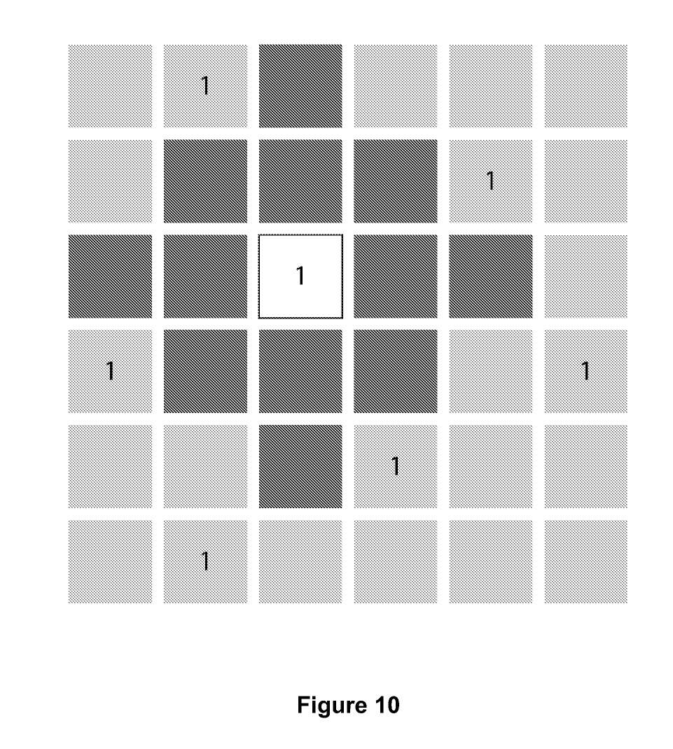

Control pins assigned to the electrodes this method in a partition allow free movement of a single droplet, i.e., the “cross constraint” is met. To demonstrate this, consider the cell which is shown in white in FIG. 10. If the cell is assigned Pin 1, the same pin cannot be assigned to the unit cells that are shaded. Otherwise, the configuration will violate the cross constraint in some cases. It can be seen that all the unit cells in the Bagua tile and its repetitions stay out of the forbidden area. Thus for each pin assigned to cells in a Bagua repetition, the cross constraint is not violated. Since this is true for any Bagua repetitions and any partition can be tiled by five copies of Bagua repetitions, the “cross constraint” is automatically met for every cell in our pin assignment method.

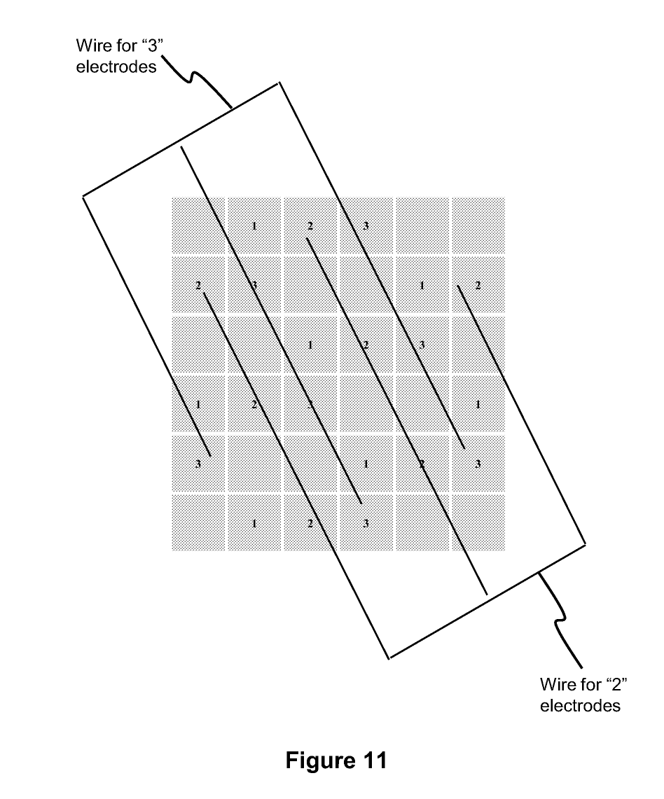

Compared to the graph coloring approach, the Connect-5 algorithm offers the important advantage that it allows wiring to be done easily on a 3-layer PCB; see FIG. 11. The graph coloring approach does not lend itself to this simple pin layout because of the likelihood of irregular vertex coloring.

### 8.1.3 Example

### Multiplexed Bioassays

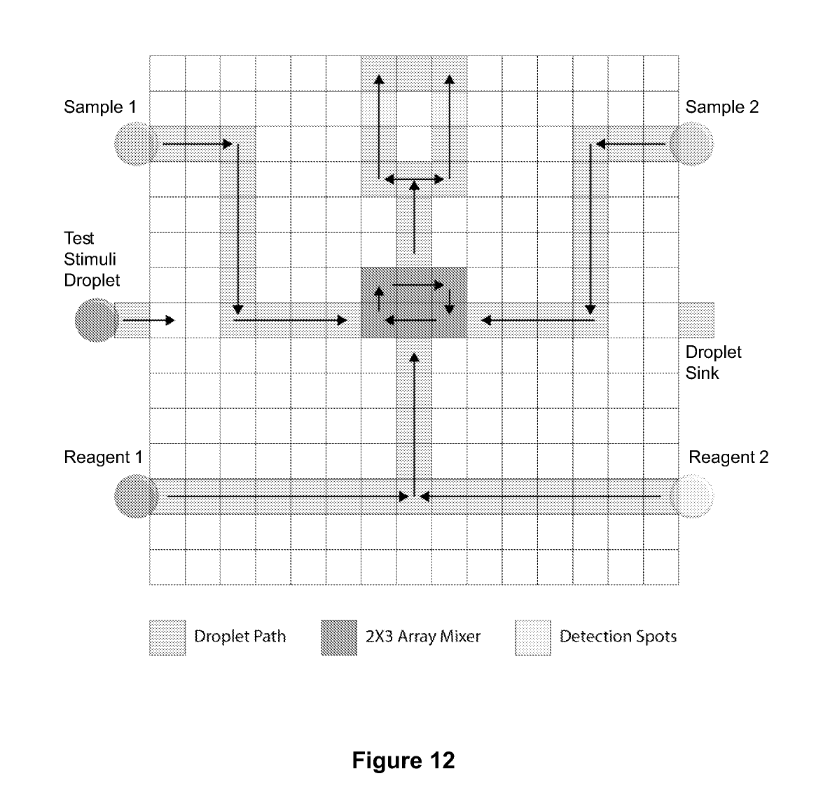

To show how partitioning and pin assignment work for pin-constrained microfluidic droplet actuators, the inventors use a real-life experiment of a multiplexed biochemical assay consisting of a glucose assay and a lactate assay based on colorimetric enzymatic reactions. These assays have been demonstrated recently [1]. The digital microfluidic droplet actuator contains a 15×15 electrode array, as shown in FIG. 12. The schedule for the set of bioassays, if a microfluidic array with 225 control pins is available, is listed in Table 2; one iteration of the multiplexed assays takes 25.8 seconds [1]. The movement of droplets is controlled using a 50 V actuation voltage with a switching frequency of 16 Hz. A depiction of the droplet paths for multiplexed glucose and lactase assays is shown in FIG. 12.

TABLE 2

| Step/Time Elapsed(s) | Operation |
|---|---|
| Step 1/0 | Sample 2 and Reagent 2 start to move towards the mixer. |
| Step 2/0.8 | Sample 2 and Reagent 2 begin to mix together and turn around in |
| | the 2 × 3-array mixer. |
| Step 3/6.0 | Sample1 and Reagent 1 start to move towards the mixer. Sample |
| | 2 and Reagent 2 continue the mixing. |
| Step 4/6.8 | Sample 2 and Reagent 2 finish the mixing and product 2 leaves |
| | the mixer to optical detection location 2. |
| | Sample 1 and Reagent 1 begin to mix in the 2 × 3-array mixer. |
| Step 5/12.8 | Sample 1 and Reagent 1 finish the mixing and product 1 leaves |
| | the mixer to the optical detection location 1. |
| | Product 2 continues the absorbance detection. |
| Step 6/19.8 | Product 2 finishes optical detection and leaves the array to the |
| | waste reservoir. |
| | Product 1 continues the absorbance detection. |
| Step 7/25.8 | Product 1 finishes optical detection and leaves the array to the |
| | waste reservoir. One procedure of the multiplexed bioassays |
| | ends. |

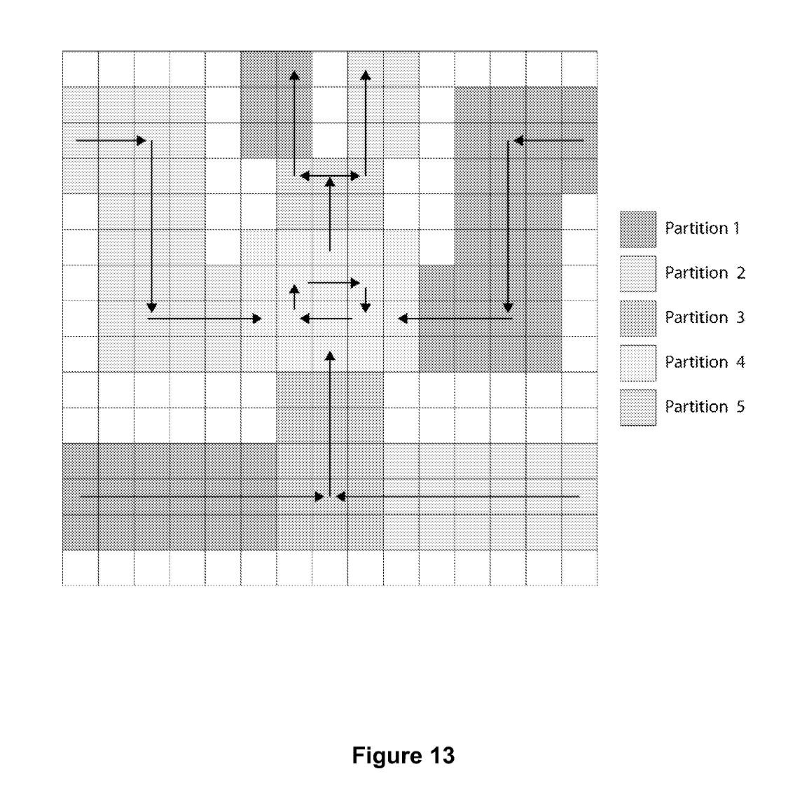

When the partitioning and pin assignment algorithm begins, six partitions are first assigned to the four droplet traces of Reactants 1, 2 and Samples 1, 2, and the two traces of the mixed samples going to Detector 1 and Detector 2. Another three partitions are assigned to the three trace-overlapping regions respectively. Next, time-span overlap is checked for the three spatial overlapping partitions (Partitions 3, 4 and 5). Since there is no temporal overlap of droplets subjected to droplet operations in both Partition 3 and Partition 5, only five pins are needed for each of them. Partition 4 is recognized as a mixer, thus only five pins are needed for it. In the next step, time span overlap is checked for all partitions pairs. The six partitions corresponding to four droplets traces and two detector paths merge into two partitions (Partition 1 and Partition 2). Finally, the Connect-5 algorithm is applied. The partitions and pin assignment results are shown in FIG. 13.

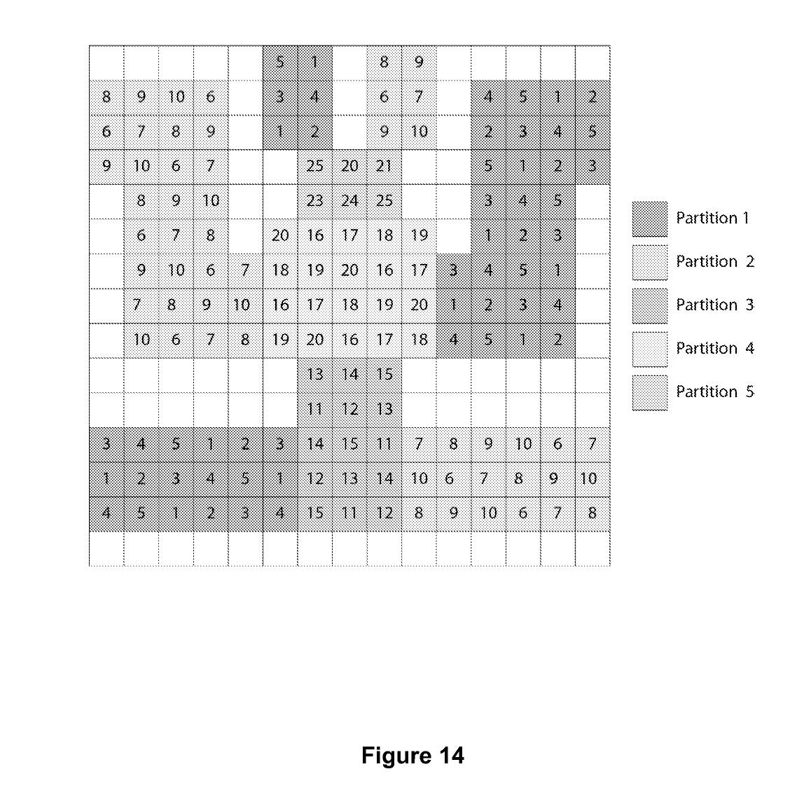

FIG. 13 shows partition assignments and FIG. 14 shows pin assignment results for the multiplexed bioassay. Blank areas are don't-care regions that can be either left unaddressed or combined with any partition.

Array partitioning and pin assignment is effective in reducing the input bandwidth, while maintaining the same throughput that is obtained for a direct-addressable array. Five partitions are satisfactory for preventing interference between multiple droplets on the array, as shown in FIG. 14. Since only five control pins are necessary for full control of a single droplet within each partition, only 25 out of the possible 225 control pins are necessary, i.e., only 11.11% of the total number of electrodes. This represents a significant reduction in input bandwidth without sacrificing throughput.

8.2 Broadcast Electrode-Addressing for Pin-Constrained Multi-Functional Droplet Actuators

The invention provides broadcast-addressing and an alternative pin-constrained design method that can be used for multifunctional droplet actuators.

8.2.1 “Don't-Cares” in Electrode-Actuation Sequences

To execute a specific bioassay, droplet routes and the schedule of operations are typically programmed into a microcontroller to drive the electrodes. Routing and scheduling information is stored in the form of (ternary) electrode activation sequences, where each bit representing the status of the electrode at a specific time-step. The status can be either “1” (activate), “0” (deactivate) or “F” (floating).

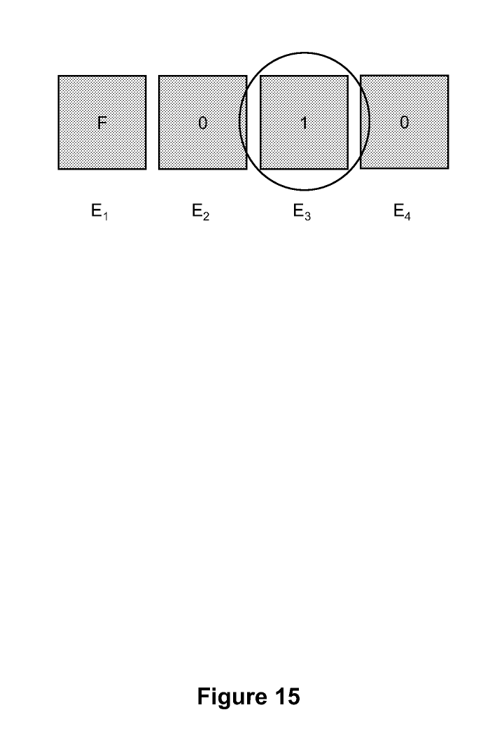

A floating signal is providing input to an electrode when it is required to be neither active nor inactive, as shown in FIG. 15. At time spot t, a droplet is to be held at electrode E₃. This electrode needs to be at high voltage (“1”), and the two adjacent electrodes E₂ and E₄ need to be deactivated (“0”). E₁ is not involved in this holding step, therefore a floating value can be assumed for it. Since the voltage on E₁ has no impact on the droplet operations for this step, E₁ can also be assigned “1” or “0”. Here, this status is represented using the symbol “x” and referred to as “don't-care”. This concept is similar to the don't-cares that arise in logic synthesis and design-for-testability during integrated circuit design.

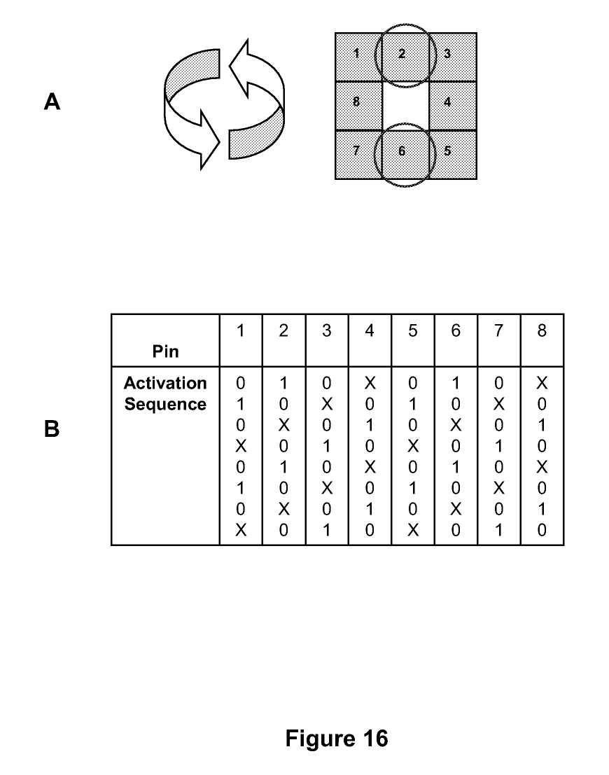

The three values “1”, “0”, and “x” are typically used to represent the electrode-activation sequences for a bioassay. An example is shown in FIGS. 16A and 16B. Two droplets are routed counterclockwise, one electrode per step, along the loop consisting of 8 electrodes. The distance between two droplets is maintained throughout to be four electrodes. Suppose that at time instant (clock cycle) t₀, the droplets rest on electrode E₂ and E₆, respectively. The activation sequence for each electrode is now calculated and listed in FIG. 16A.

In FIG. 16B, each sequence contains several don't-care terms, which can be replaced by “1” or “0”. By carefully replacing these don't-care terms, the two activation sequences corresponding to E₁ and E₃ can be made identical. For example, all the don't-cares in these activation sequences can be mapped to “1”. Such sequences are referred to as compatible sequences. Compatible sequences can be generated from a single signal source. Therefore, the corresponding electrodes E₁ and E₃ can be connected to a single control pin.

8.2.2 Optimization Based on Clique Partitioning in Graphs

One aspect of the invention is focused on reducing the number of control pins by connecting together electrodes with mutually-compatible activation sequences, and addressing them using a single control pin. Therefore, the resulting electrode-access method is referred to as broadcast-addressing. The electrodes are first partitioned into groups. For all the electrodes in any group, the corresponding activation sequences must be pairwise-compatible. The typical goal is to find an optimal partition that leads to the minimum number of groups, which in turn yields the minimum number of control pins.

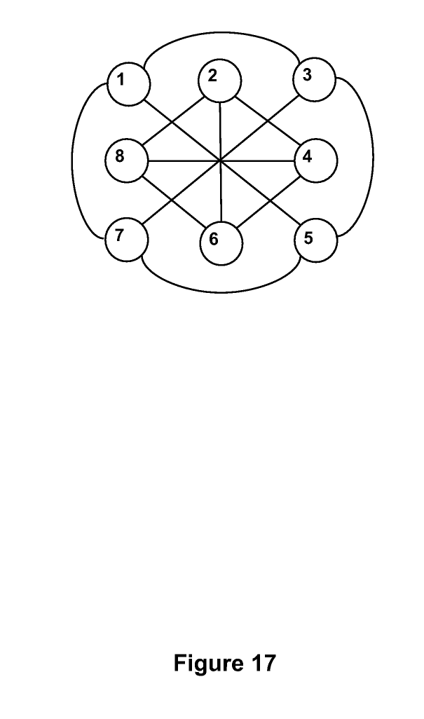

The problem of finding the minimum number of groups can be easily mapped to the clique-partitioning problem from graph theory [31]. The example in FIGS. 16A and 16B can be to illustrate this mapping. Based on the activation-sequence table, an undirected graph, referred to as electrode-activation graph, is constructed; see FIG. 17. Each node in the graph represents an activation sequence for an electrode. An edge in the graph between a pair of nodes indicates that their corresponding activation sequences are compatible. For example, nodes 1 and 3, which represent the activation sequences for electrode E₁ and E₃, respectively, are connected by an edge because the activation sequences can be converted to a single sequence “01010101” by replacing all the don't-care terms with “1”s.

A clique in a graph is defined as a complete subgraph, i.e., any two nodes in this subgraph are connected by an edge [31]. Clique partitioning refers to the problem of dividing the set of nodes into non-overlapping subsets such that the subgraph induced by each subset of nodes is a clique. A minimal clique partition is one that covers the nodes in the graph with a minimum number of non-overlapping cliques. The grouping of droplets as discussed above is equivalent to the clique-partitioning problem. A minimal clique partition for this example is given by {1,3,5,7}, {2,4,6,8}. Even though the general clique partitioning problem is known to be NP-hard [31], a number of heuristics are available in the literature to solve it in an efficient manner.

After an efficient partitioning of electrodes is derived, all the electrodes in a group can be addressed using a single control pin. A common activation sequence compatible to all the individual sequences in each group is calculated and used as the input sequence for the control pin. In the above example, electrodes E₂, E₄, E₆, E₈ are connected and they share the common activation sequence of {10101010}. Since a common activation sequence is broadcasted to several electrodes, this addressing method can be referred to as “broadcast-addressing”.

The complete steps in an exemplary embodiment of broadcast-addressing as contemplated herein are typically as follows:

1. Obtain droplet-routing information from the droplet actuator synthesis results and calculate the control-signal sequence for each control pin. The control-signal sequence consists of the values 1 (activated), 0 (deactivated), and x (don't-care).

2. Draw an undirected graph representing the relationship between control-signal sequences. For every pair of electrode-activation sequence, if one sequence can be derived from the other by simply changing x's to 1's/0's, then draw an edge between the nodes representing them.

3. Apply clique partitioning to minimize the number of independent control signals.

4. Group and connect the control lines that are in the same clique.

By using this broadcast-addressing method, the input bandwidth for the microfluidic droplet actuator can be significantly reduced. For the example in FIGS. 16A and 16B, instead of using eight independent control pins to address the electrode loop, broadcast-addressing only needs two control pins.

Another advantage of the broadcast-addressing method is that it provides maximum freedom of droplet movement. It does not change the schedule of operations or the droplet-routing pathways for the target bioassay; therefore, bioassays can be executed as fast as on a direct-addressing-based droplet actuator. Compared to the array-partitioning-based method [20], broadcast-addressing does not need to limit the number of concurrent droplet movements to get fewer partitions. The method disclosed herein also reduces assay operation time compared to cross-referencing [2]; the latter typically requires several sub-steps for a set of droplet operations that can be carried out concurrently in a direct-addressing-based droplet actuator. These advantages are quantitatively evaluated using a real droplet actuator example as discussed herein below in section 8.2.4.

8.2.3 Broadcast-Addressing for Multifunctional Droplet Actuators

Broadcast-addressing can also be typically applied to multifunctional droplet actuators. For each target bioassay, droplet routing and schedule information are collected and activation sequences are calculated. Next, for each electrode, the activation sequences from the different assays can be merged and a collective activation sequence obtained. Note that the compatibility of activation sequences is typically independent of the ordering of the sequences. Therefore, the merging of activation sequences can typically be carried out in any arbitrarily-chosen order.

Once the collective activation sequences are derived, the same steps as described herein are carried out to derive the electrode partitions and the wiring (connection of input pins to electrodes) plan.

Note that the longer the activation sequences, the more specified entries, i.e., “1” and “0” exist, and less compatibility is observed. Therefore, multi-functionality may necessitate a larger number of input control pins for the broadcast-addressing method disclosed herein. This trade-off is evaluated herein below.

8.2.4 Experimental Results

In exemplary embodiments, the broadcast-addressing method disclosed herein is evaluated by using pin-constrained design of droplet actuators for a multiplexed immunoassay, a representative protein assay, and the polymerase chain reaction (PCR) procedure.

Each assay is first mapped to a 15×15 electrode array controlled using the direct-addressing scheme. Next, the broadcast-addressing method disclosed herein is used to reduce the number of control pins.

8.2.5 Multiplexed Assay

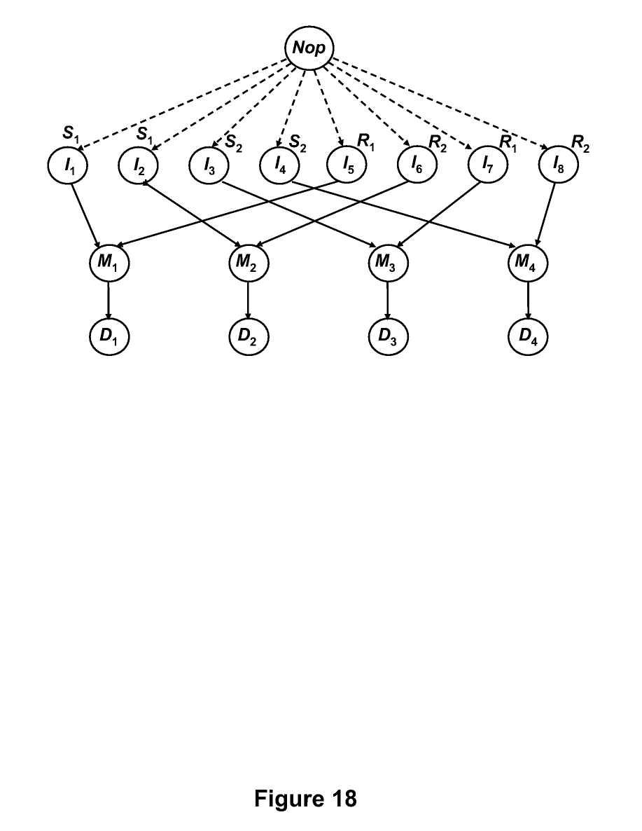

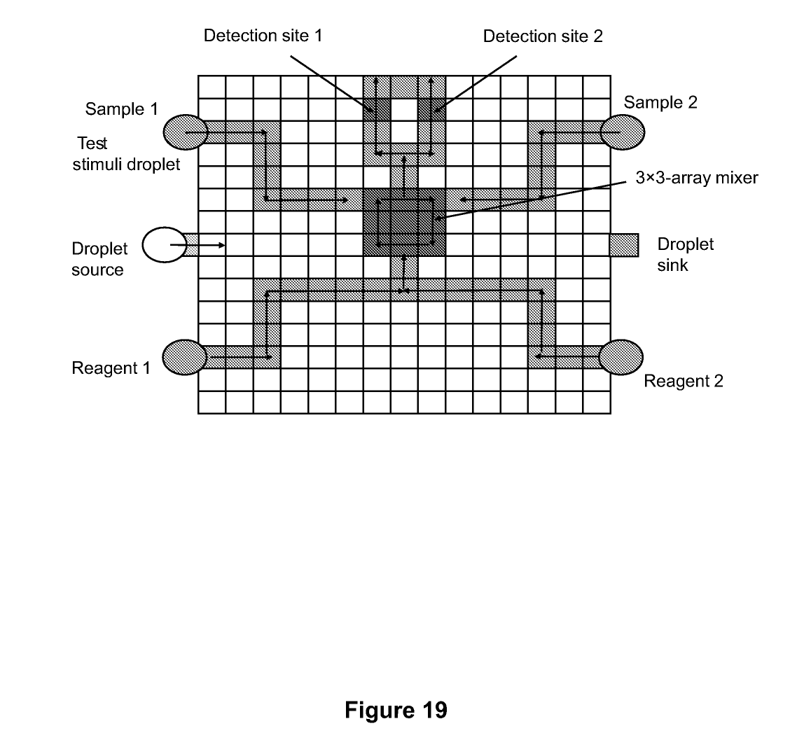

A recently demonstrated multiplexed biochemical assay is first mapped, which consists of a glucose assay and a lactate assay based on colorimetric enzymatic reactions, on to the array. FIG. 18 shows the flowchart for the multiplexed assays in the form of a sequencing graph [16]. For each sample or reagent, two droplets are dispensed into the array. Four pairs of droplets, i.e., {S₁, R₁}, {S₁, R₂}, {S₂, R₁}, {S₂, R₂}, are routed together in sequence for the mixing operation. Mixed droplets are finally routed to the detection site for analysis. In [1], the multiplexed bioassays were mapped to a digital microfluidic platform containing a 15×15 array, as shown in FIG. 19. A depiction of the droplet pathways for multiplexed glucose and lactase assays is given in FIG. 19.

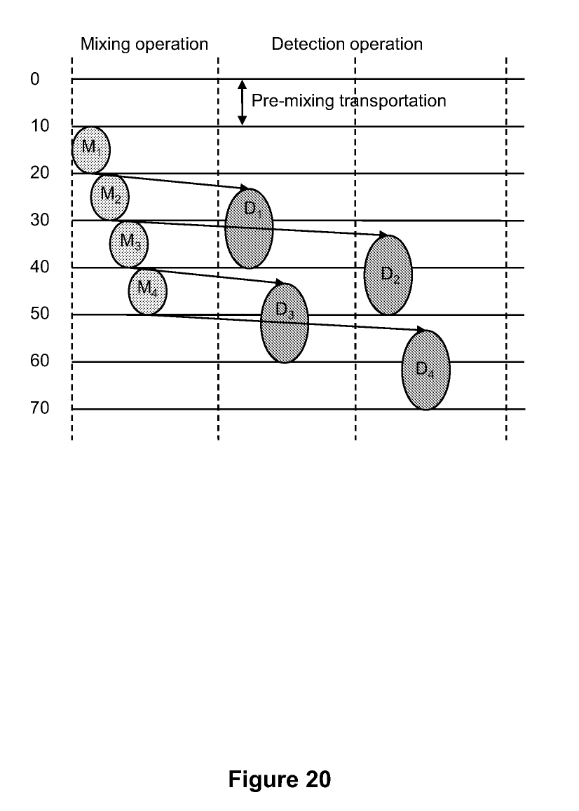

In the multiplexed assay, eight droplets (two droplets from each sample/reagent) are dispensed and routed to the mixer located at the center. Next, four mixing and detection operations are carried out in a pipeline manner following the schedule shown in FIG. 20. The droplets are assumed to be transported at the rate of 1 electrode/second, i.e., 1 Hz.

Next, the broadcast-addressing method disclosed herein is applied to the above droplet actuator layout. As shown in FIG. 19, multiplexed assay droplet actuator utilizes 59 electrodes. The electrode activation sequences are calculated based on the scheduling and routing result presented herein. A fragment of the activation sequences is listed in Table 3.

TABLE 3
A fragment of the activation sequences for multiplexed assay.

| Elec- | | | | | | | | | | | | | |
|---|---|---|---|---|---|---|---|---|---|---|---|---|---|
| trode # | | | | | | | | | | | | | |
| (7~20) | Activation Sequences(0 s~13 s) | | | | | | | | | | | | |
| . | . | . | . | . | . | . | . | . | . | . | . | . | . |
| . | . | . | . | . | . | . | . | . | . | . | . | . | . |
| . | . | . | . | . | . | . | . | . | . | . | . | . | . |
| 7 | 1 | 0 | x | x | x | 0 | 1 | 0 | x | x | x | 0 | . . . |
| 8 | 0 | 1 | 0 | x | x | x | 0 | 1 | 0 | x | x | x | . . . |
| 9 | x | 0 | 1 | 0 | x | x | x | 0 | 1 | 0 | x | x | . . . |
| 10 | x | x | x | x | x | x | x | x | x | x | x | x | . . . |
| 12 | x | x | x | x | x | x | x | x | x | x | x | x | . . . |
| 13 | x | 0 | 1 | 0 | x | x | x | 0 | 1 | 0 | x | x | . . . |
| 14 | 0 | 1 | 0 | x | x | x | 0 | 1 | 0 | x | x | x | . . . |
| 15 | 1 | 0 | x | x | x | 0 | 1 | 0 | x | x | x | 0 | . . . |
| 16 | x | x | 0 | 1 | 0 | x | x | x | 0 | 1 | 0 | x | . . . |
| . | . | . | . | . | . | . | . | . | . | . | . | . | . |
| . | . | . | . | . | . | . | . | . | . | . | . | . | . |
| . | . | . | . | . | . | . | . | . | . | . | . | . | . |

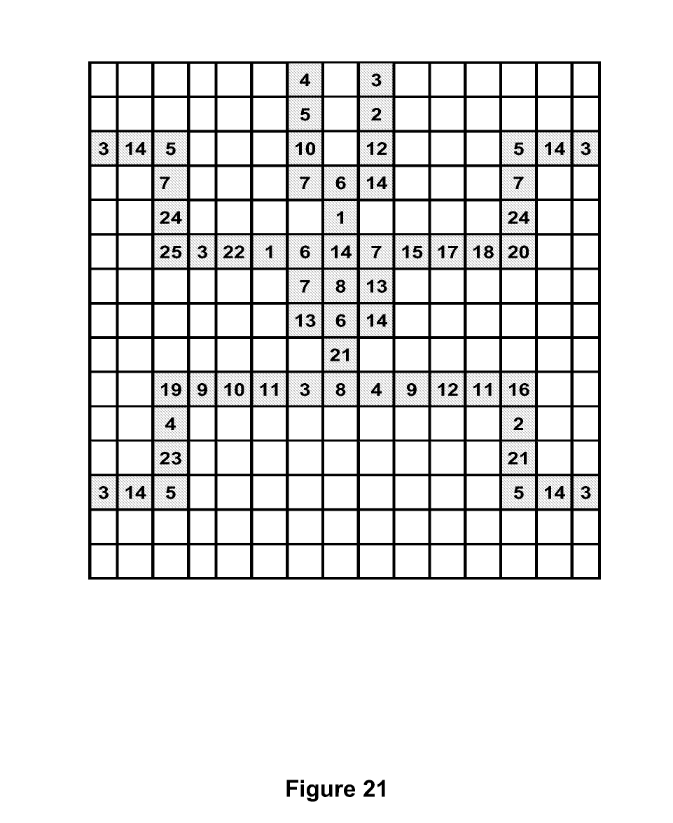

Next, the clique-partitioning-based broadcast-addressing method is used to generate the electrode connections and the pin-assignment plan. The results are shown in FIG. 21. The pins assigned to the electrodes are shown in the corresponding boxes.

In FIG. 21, the number of control pins is reduced from 59 to 25, almost a 60% reduction compared to direct-addressing method. Due to considerable reduction in wiring complexity, fabrication cost is reduced significantly. There is no increase in the assay time compared to a direct-addressing droplet actuator that uses 59 electrodes.

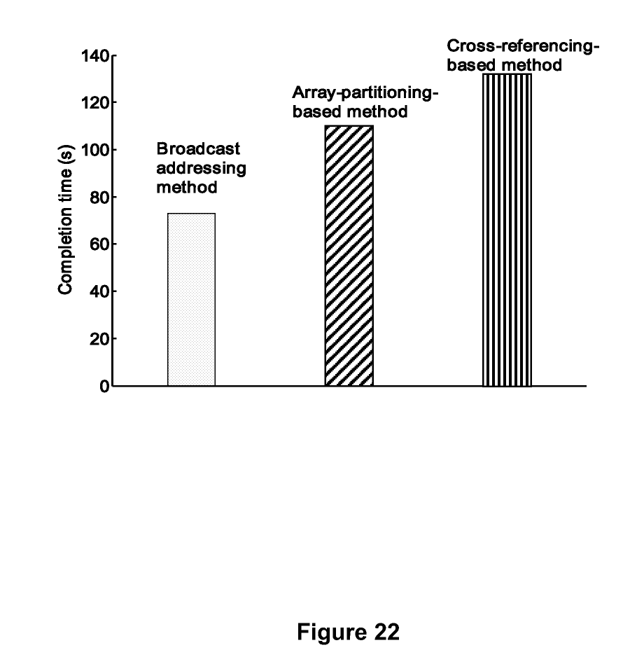

The array-partitioning-based method and cross-reference-based method also lead to a significant reduction in number of control pins, but at the expense of higher assay completion times. The results are shown in FIG. 22. With broadcast-addressing, an assay completion time of 73 s was obtained. The array-partitioning-based method requires the same number of control pins but a longer completion time of 110 s. The cross-referencing-based method requires 30 control pins but with a completion time of 132 s.

8.2.6 Polymerase Chain Reaction (PCR)

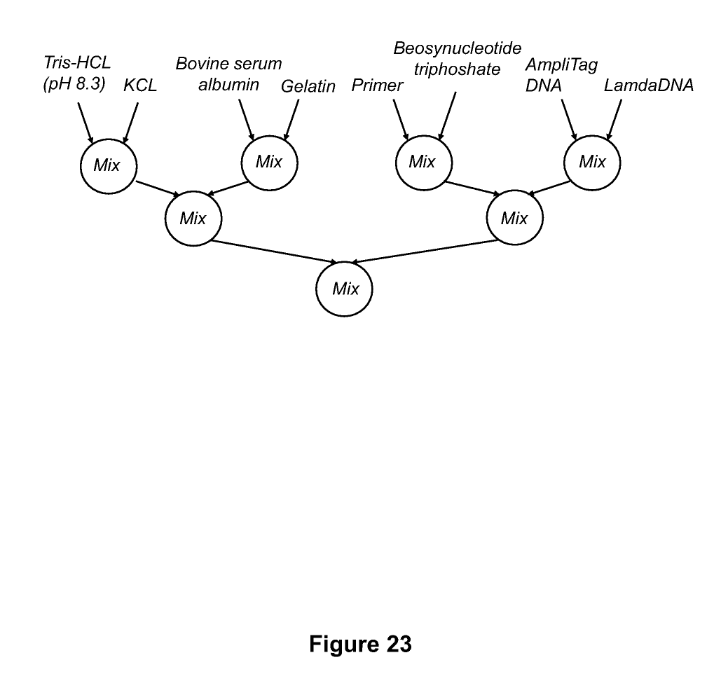

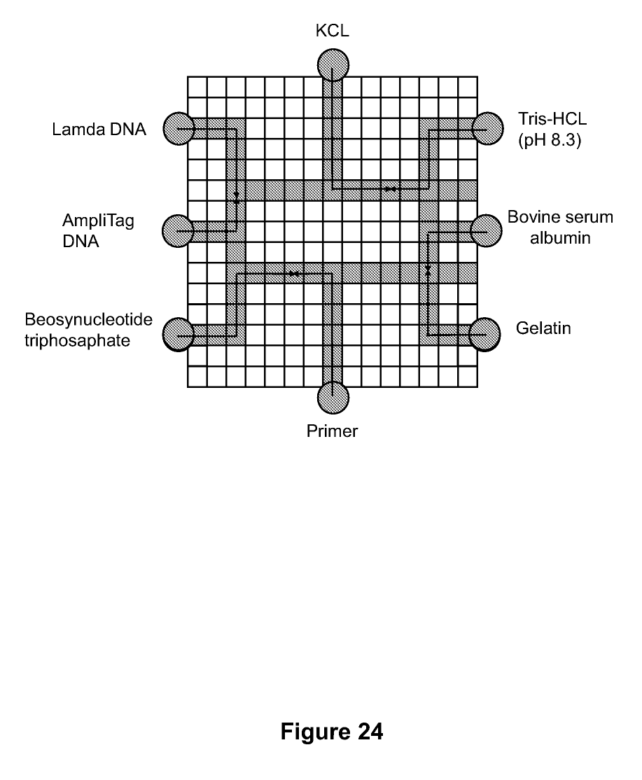

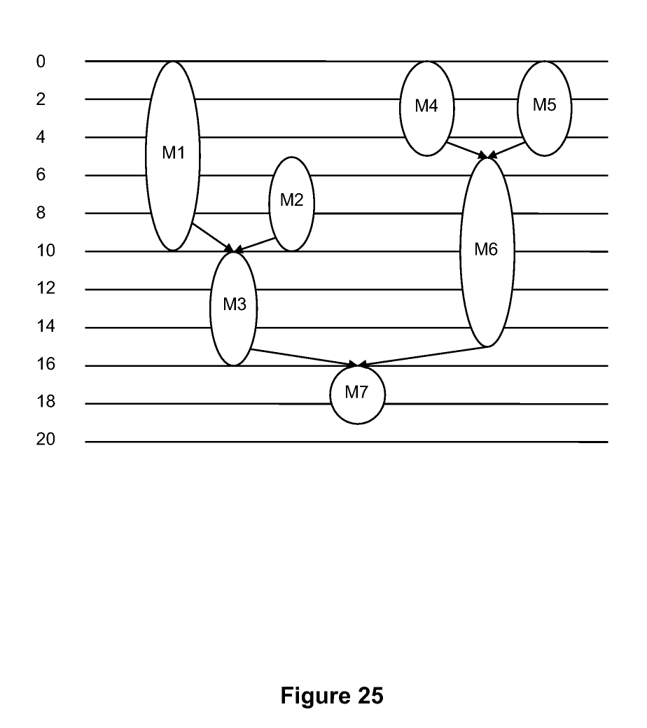

For the second assay, the mixing stages of the PCR is used. These stages are used for rapid enzymatic amplification of specific DNA strands. Recently, the feasibility of performing droplet-based PCR on digital microfluidics-based droplet actuators has been successfully demonstrated [21]. Its assay protocol can be modeled by a sequencing graph, as shown in FIG. 23. Mapping the protocol on to the array, the droplet actuator layout and schedule is obtained as shown in FIG. 24 and FIG. 25, respectively.

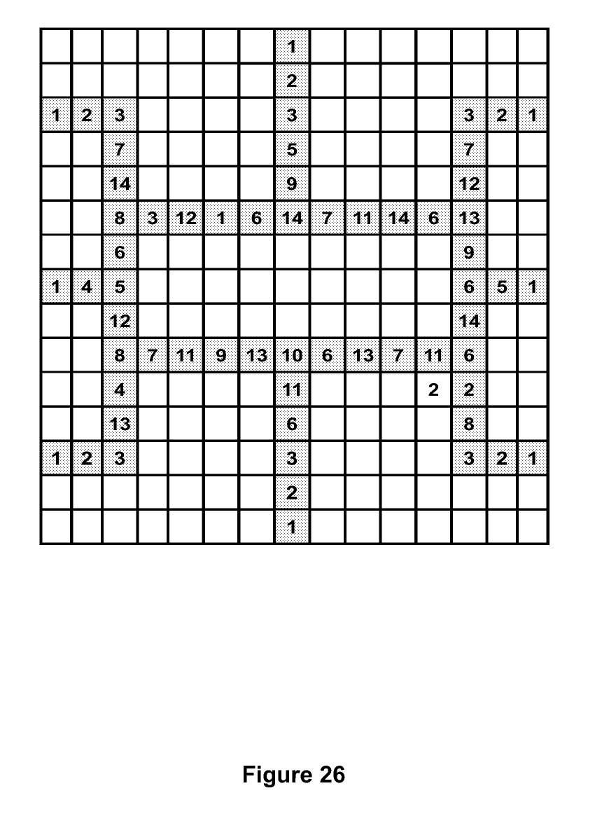

Assuming a direct-addressing scheme, the layout in FIG. 24 requires 62 control pins. However, using the broadcast-addressing method disclosed herein, we reduce the number of control pins to 14. The pin-constrained layout for the PCR droplet actuator is shown in FIG. 26.

8.2.7 Protein Dilution

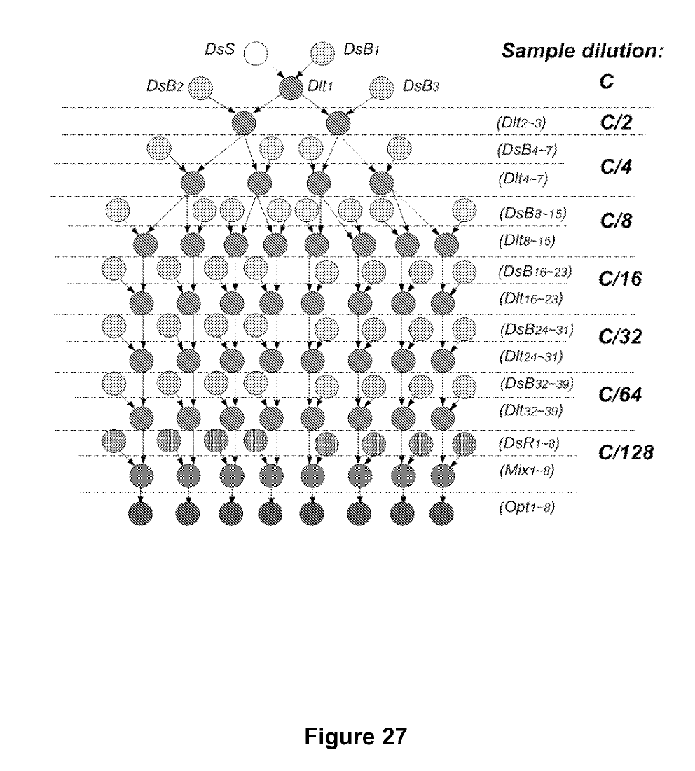

The third assay that is considered as an example consists of the dilution steps in a real-life protein assay. Its assay protocol can be modeled by a sequencing graph, as shown in FIG. 27. The feasibility of performing a colorimetric protein assay on a digital microfluidic droplet actuator has been successfully demonstrated [16]. Based on the Bradford reaction [16], the protocol for a generic droplet-based colorimetric protein assay is typically as follows. First, a droplet of the sample, such as serum or some other physiological fluid containing protein, is generated and dispensed into the droplet actuator. Buffer droplets, such as 1M NaOH solution, are then introduced to dilute the sample to obtain a desired dilution factor (DF). This on-droplet actuator dilution is performed using multiple hierarchies of binary mixing/splitting phases, referred to as the interpolating serial dilution method [4]. The mixing of a sample droplet of protein concentration C and a unit buffer droplet results in a droplet with twice the unit volume, and concentration C/2.

Splitting this large droplet results in two unit-volume droplets of concentration C/2 each. Continuing this step in a recursive manner using diluted droplets as samples, an exponential dilution factor of DF=2^{N} can be obtained in N steps. After dilution, droplets of reagents, such as Coomassie brilliant blue G-250 dye, are dispensed into the droplet actuator, and they mix with the diluted sample droplets. Next the mixed droplet is transported to a transparent electrode, where an optical detector (e.g., a LED-photodiode setup) is integrated. The protein concentration can be measured from the absorbance of the products of this colorimetric reaction using a rate kinetic method.

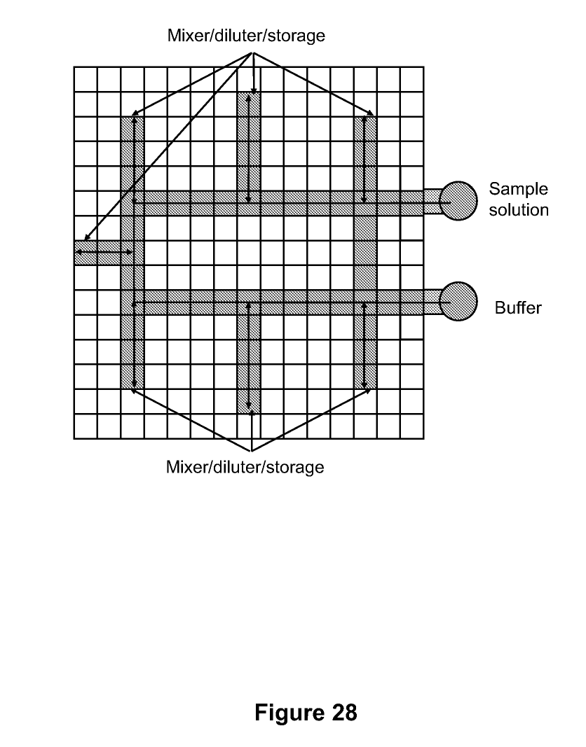

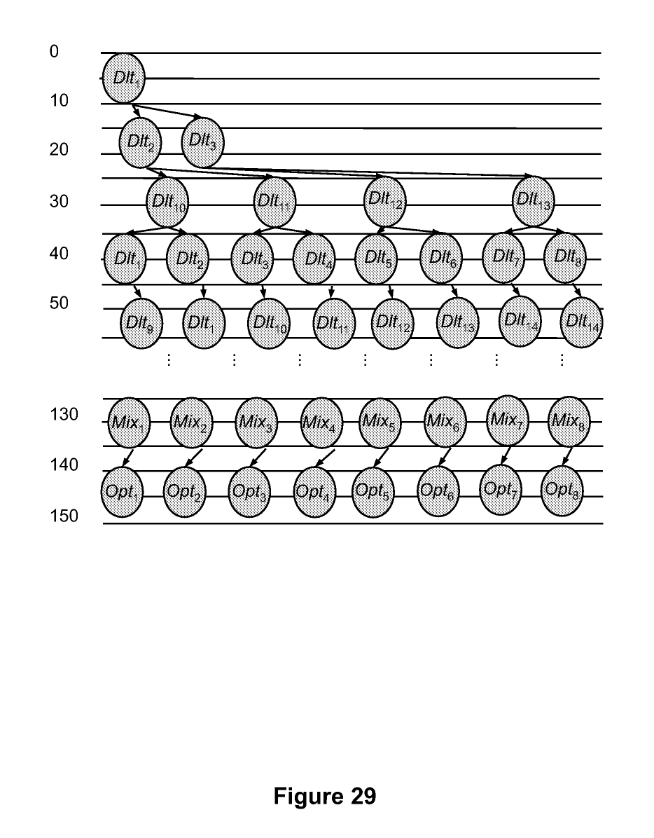

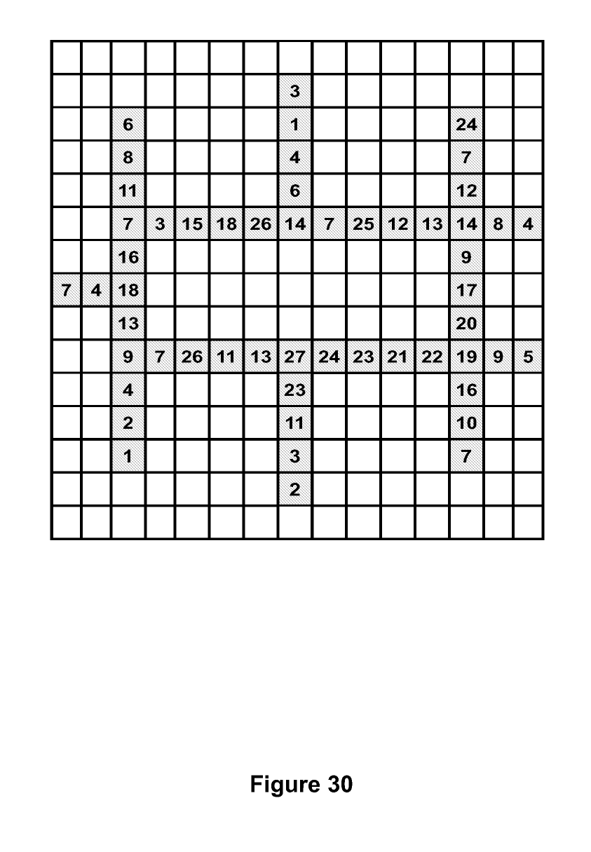

The protein assay is mapped to the 15×15 array. FIG. 28 shows the droplet actuator layout and FIG. 29 illustrates the schedule for this protocol. In FIG. 28, 52 electrodes are used in the droplet actuator layout. This number is reduced to only 27 after the broadcast-addressing method is applied; see FIG. 30.

8.2.8 Broadcast-Addressing for a Multi-Functional Droplet Actuator

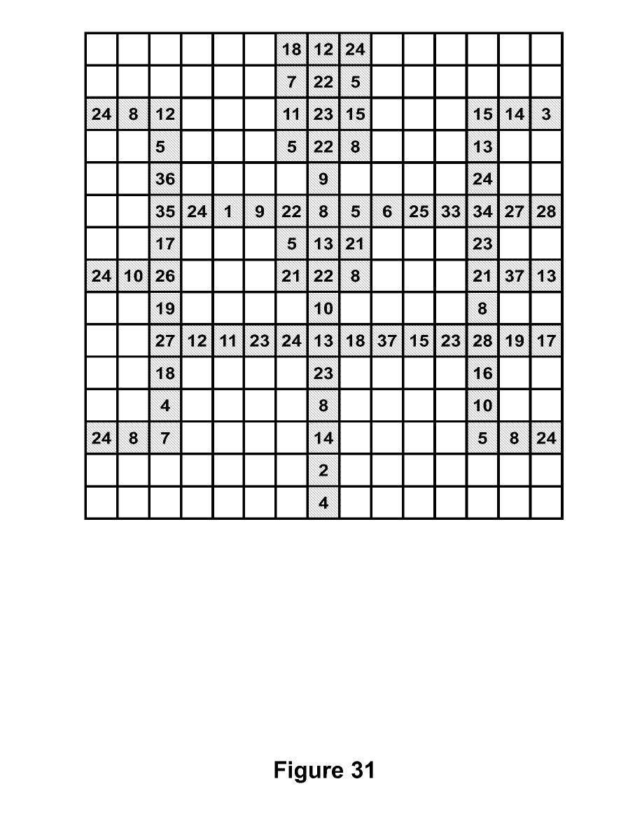

Finally, the performance of the method disclosed herein for multi-functional droplet actuator design is evaluated. Here, a multi-functional droplet actuator is designed that can execute all the three assays described in the previous subsections. The pin-assignment for the multi-functional droplet actuator can be obtained by combining the droplet actuator layouts for the three different assays, see FIG. 31. Note that only 81 electrodes on the 15×15 array are used in this layout and thereby need to be addressed.

Next, the addressing problem for the multi-functional droplet actuator is considered. The activation sequences for the PCR assay and protein assay are determined and combined with that from the multiplexed assay. The broadcast-addressing method is carried out and it generates a droplet actuator layout with only 38 control pins.

The addition of two assays to the droplet actuator for multiplexed assay and 22 (81−59=22) new electrodes leads to only 13 extra control pins. These results highlight the scalability attribute of the design method disclosed herein.

8.3 Cross-Referencing-Based Droplet Operations Method for High-Throughput and Pin-Constrained Digital Microfluidic Arrays

The invention relates to performing droplet operations on multiple droplets based on digital microfluidic droplet actuators that use cross-referencing to address the electrodes, such as through interference-free droplet manipulation based on destination-cell categorization.

### 8.3.1 Electrode Interference

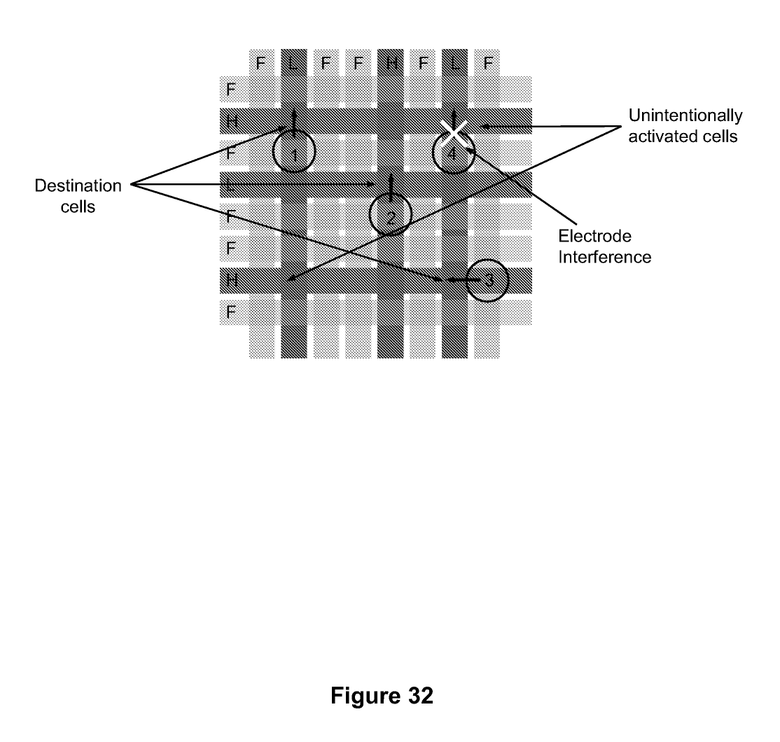

For performing droplet operations on multiple droplets concurrently on a cross-referencing-based droplet actuator, multiple row and column pins are typically selected to activate the destination cells, i.e., cells to which the droplets are supposed to move. However, the selected row and column pins may also result in the activation of cells other than the intended droplet destinations. An example is shown in FIG. 32. The goal here is to route Droplets 1, 2, and 3 simultaneously to their destination cells. Droplet 4 is supposed to remain in its current location. However, two additional cells are activated unintentionally when the activation voltage is applied to the row and column pins corresponding to the destination cells. As a result, Droplet 4 is unintentionally moved one cell up (along the Y-direction). This phenomenon is referred to as electrode interference.

### 8.3.2 Destination-Cell Categorization

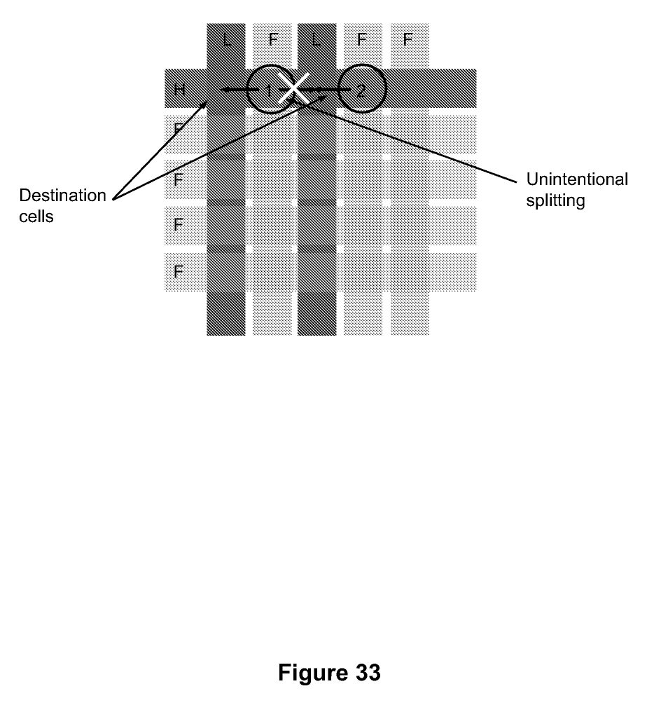

As shown in FIG. 32, the concurrent droplet operations of multiple droplets must be carried out without introducing any electrode interference. Here, a solution based on destination-cell categorization is contemplated. Note that the problem highlighted in FIG. 32 can be avoided if the destination cells of the droplets being moved simultaneously reside on the same column or row. However, electrode interference may still occur within the same column or row, as shown in FIG. 33.

Referring to FIG. 33, suppose Droplet 1 and Droplet 2 are both moved one cell to the left at the same time. Even though no additional cells are activated unintentionally, Droplet 1 undergoes unintentional splitting in this situation. Fortunately, this problem can be avoided where Pᵢ(t) is the position of droplet i at time t and Pⱼ(t) is the position of droplet j at time t.

The fluidic constraints avoid unintentional fluidic operations that arise due to the overlapping of droplets over adjacent electrodes. Thus they apply to both direct-addressing-based and cross-referencing-based droplet actuators. In FIG. 33, the intended multiple droplet operations violates the constraint |Pᵢ(t+1)−Pⱼ(t)|≧2. If the fluidic constraints are satisfied at all times, it is safe to carry out concurrent droplet operations of multiple droplets whose destination cells are accessed by the same column or row in practice by satisfying the fluidic constraints described in [21]. These constraints are given by the following set of inequalities: (i) |Pᵢ(t)−Pⱼ(t)|≧2; (ii) |Pᵢ(t+1)−Pⱼ(t)|≧2; (iii) |Pᵢ(t)−Pⱼ(t+1)|≧2; (iv) |Pᵢ(t+1)−Pⱼ(t+1)|≧2.

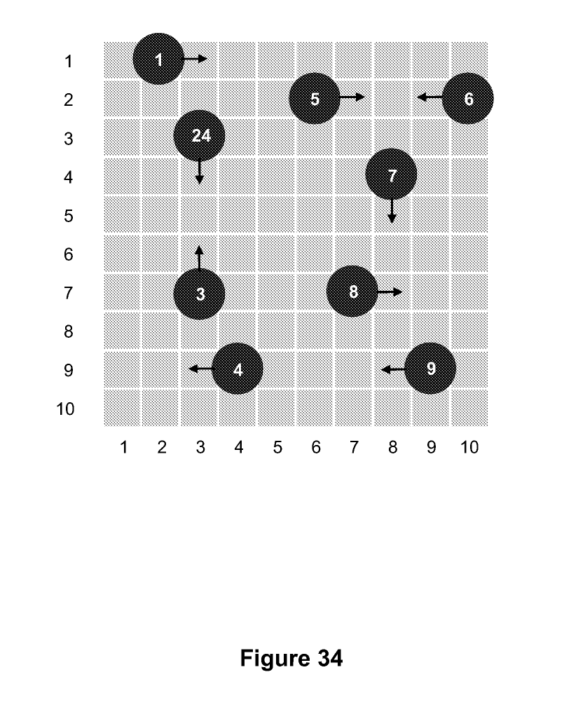

On the basis of the above observations, the droplets that can be moved simultaneously are considered as part of the bioassay, and are placed in different groups. A group consists of droplets whose destination cells share the same column or row. An example is shown in FIG. 34. A total of nine droplets are needed to be moved on a 10×10 array. As discussed above, the droplet movements are grouped according to their destination cells. For example, Droplets 4 and 9 form a group since the destination cells in both cases reside on Row 2. Similarly Droplets 1, 2, and 3 are placed in the same group since they are all moving to Column 3. Following this grouping process, we finally get four groups of droplets, i.e., {4,9}, {1,2,3}, {5,6}, {7,8}.

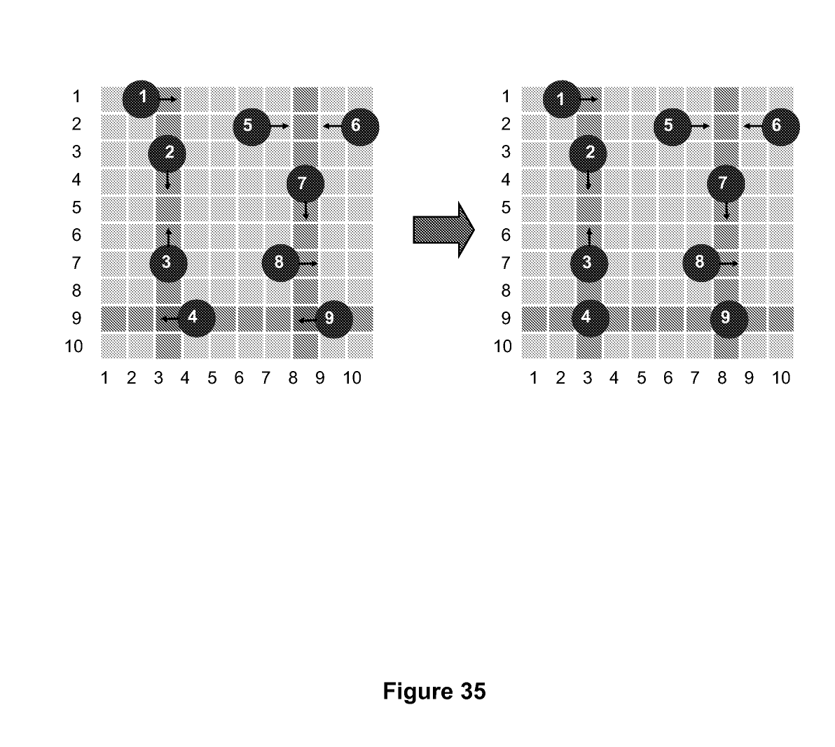

In this way, performing droplet operations on multiple droplets is ordered in time; droplets in the same group can be moved simultaneously without electrode interference, but the movements for the different groups must be sequential. For example, droplet movements for the group {4,9} in FIG. 34 can be carried simultaneously, as shown in FIG. 35. Droplet movements are carried out one group after another until all the droplet movements are completed.

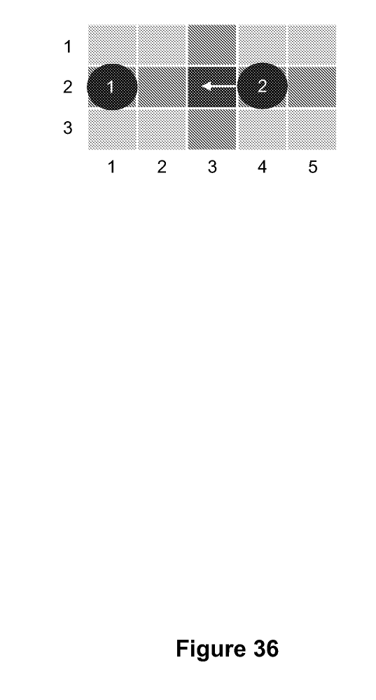

Note that the ordering of droplet movements based only on the above grouping strategy can cause electrode interference and inadvertent mixing. An example is shown in FIG. 36. The movement of Droplet 2 alone to the left by activating Column 3 will not influence Droplet 1. Similarly, the movement of Droplet 1 alone to the right by activating Column 2 will not influence Droplet 2. However, if these two droplets are moved concurrently, as determined by the grouping procedure, by the activation of (Column 2, Row 2) and (Column 3, Row 2), they mix at (3,2). However, manipulations of this type violate the fluidic constraint given by |Pᵢ(t+1)−Pⱼ(t+1)|≧2. Therefore, such problems can be avoided if the grouping procedure incorporates the fluidic constraints.

Although the grouping of droplets based on destination cells reduces the number of droplets that can be simultaneously moved, this approach provides more concurrency than the baseline method of moving one droplet at a time. Compared to direct-addressing, an order of magnitude reduction in the number of control pins is obtained. Simulation results in section 8.3.5 show that there is only a small increase in the bioassay processing time compared to direct-addressing.

### 8.3.3 Graph-Theoretic Model and Clique Partitioning

The basic idea of performing droplet operations on multiple droplets based on destination-cell categorization has thus far been introduced, and the droplets in each group have been shown to move simultaneously. Assuming that each step takes constant processing time, the total completion time for a set of droplet movement operations is typically determined by the number of groups derived from the categorization of destination cells. Note however that the grouping need not be unique. For instance, in the example of FIG. 34, four groups can be formed, i.e., {4,9}, {1,2,3}, {5,6} and {7,8}. However, {1,2,3,4}, {5,6}, {7,8,9} is also a valid grouping of the droplets. The latter grouping is preferable because three groups allow more concurrency, and therefore lower bioassay completion time.

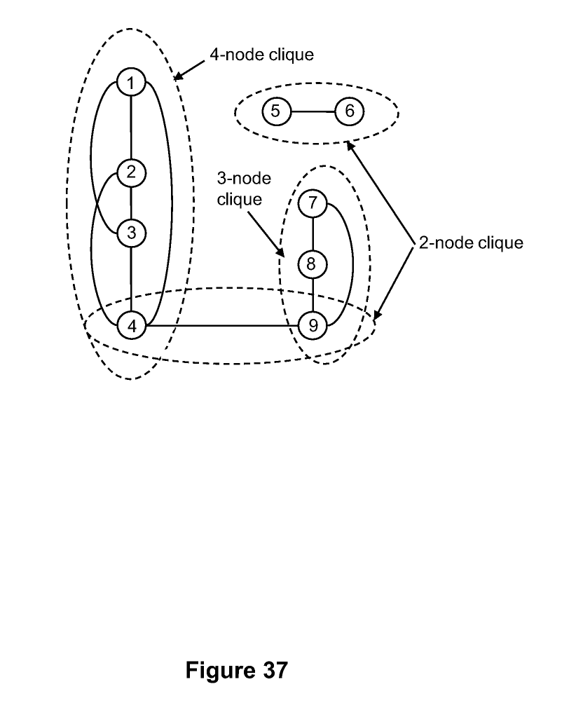

The problem of finding the minimum number of groups can be directly mapped to the clique partitioning problem from graph theory [31]. To illustrate this mapping, the droplet operations problem that is defined in FIG. 34 is used. Based on the destinations of the droplets, an undirected graph, referred to as the droplet movement graph, is constructed for each time-step; see FIG. 37. Each node in the droplet movement graph represents a droplet. An edge in the graph between a pair of nodes indicates that the destination cells for the two droplets either share a row or a column. For example, Nodes 1 and 2, which represent Droplet 1 and Droplet 2, respectively, are connected by an edge because the destination cells for these droplets are accessed using Column 3 in the array. Similarly, Nodes 4 and 9 are connected by an edge because the corresponding destination cells are addressed using the same row.

A clique in a graph is defined as a complete subgraph, i.e., any two nodes in this subgraph are connected by an edge [31]. Clique partitioning refers to the problem of dividing the nodes into overlapping subsets such that the subgraph induced by each subset of nodes is a clique. A minimal clique partition is one that covers the nodes in the graph with a minimum number of non-overlapping cliques. The grouping of droplets as discussed above is equivalent to the clique partitioning problem. The categorization of destination cells using the grouping of droplets is equivalent to the problem of determining a minimal clique partition. Cliques of different sizes for a given droplet movement graph are shown in FIG. 37. A minimal clique partition here is given by {1,2,3,4}, {5,6}, {7,8,9}, which corresponds to the groups derived herein. Even though the general clique partitioning problem is known to be NP-hard [32], a number of heuristics are available in the literature to solve it in an efficient manner.

### 8.3.4 Algorithm for Droplet Grouping

Next, a greedy algorithm is described to determine a (minimal) clique partition for the droplet movement graph (DMG). The algorithm determines cliques for the DMG in an iterative manner. The largest clique is first determined and then nodes and edges corresponding to this clique are deleted form the graph. Next, the clique searching procedure is applied to the reduced graph. The algorithm terminates when all the nodes in the DMG have been deleted, i.e., an empty graph is obtained. The computational complexity of this problem for the DMG is linear in the number of rows/columns. It is understood that the cliques can only typically be formed among nodes sharing the same row or column. Therefore, the largest clique can be determined by scanning the columns and rows of the array. Thus a maximum of only MM iterations are needed for the droplet movement graph derived from an N×M array.

Note that even though in each step of the above algorithm, the largest clique and the associated destination cells are deleted, the absence of the corresponding destination cells does not lead to any added complexity for droplet movement. This is because the droplet movements involving these destination cells are incorporated in the clique determined at this step. Therefore, when the algorithm terminates with an empty graph, all droplet movements have been processed without any electrode interference.

The typical steps of the complete procedure to determine the order of droplet movements in an exemplary embodiment can be stated as follows:

1. Obtain the required droplet movements (from a synthesis tool such as [33]), and organize these movements in the form of snapshots corresponding to different time-steps. The fluidic constraints described herein typically need to be satisfied for each snapshot.

2. Compare consecutive snapshots to determine the destination cells for the droplets.

3. Scan each row and each column to find the row/column with the largest set of destination cells. The destination cells thus determined forms a group of droplets that can be simultaneously moved. If no row/column contains more than one destination cells, set the flag END to 1.

4. If END=1, process the remaining movements in multiple steps, but with two droplets at each step. Else carry out the droplet movements indicated by Step 3.

5. Check if all the movements in the snapshot have been processed. If the check yields a negative outcome, repeat Step 3.

6. Check whether all the snapshots are processed. If not, get the next snapshot and repeat Step 2, else terminate the procedure.

### 8.3.5 Evaluation and Simulation Results

As an exemplary example, Monte-Carlo simulation and a set of multiplexed bioassays were used to evaluate the method disclosed herein.

### 8.3.6 Monte Carlo Simulation

Monte-Carlo simulation can be used to evaluate the effectiveness of the droplet movement approach disclosed herein. Digital microfluidic arrays of size N×N, (N=25, 50, 75) are considered herein. For each array, 1000 simulated droplet movement plans are considered. Each droplet movement plan is defined by a starting snapshot and destination snapshot. The starting snapshot is generated by injecting a droplet in the array with probability k, referred to as the droplet injection probability (DIP). A special check is incorporated in the generation process to avoid the violation of fluidic constraints. Results derived from this process can be viewed as snapshots of droplets moving around the droplet actuator. Each droplet movement plan is provided as input to the method disclosed herein and the number of steps required for droplet movement is calculated. One-at-a-time droplet movement is also considered and the results are recorded for the purpose of comparison.

To evaluate the method disclosed herein, the parameter “number-of-steps-ratio” (NSR) is introduced, defined by the equation NSR=Nₚ/Nₒ, where Nₚ (Nₒ) is the number of movement steps for the method (one-at-a-time baseline method). Small values of NSR are clearly desirable. The NSR values are calculated for different array sizes and the results are as shown in Table 4.

As shown in Table 4, regardless of DIP value, the NSR decreases with array size. This shows that the method disclosed herein is typically more efficient for performing droplet operations on multiple droplets concurrently on large-scale digital microfluidic arrays. For a given array size, the method disclosed herein achieves lower NSR values for higher values of DIP. Thus we see that compared to the one-at-a-time scheme, droplets can be manipulated more efficiently for high-throughput droplet actuators with higher concurrency in droplet actuator operations.

TABLE 4
Results for Monte Carlo
simulation, sample size = 1000

| DIP | Array Size | NSR |
|---|---|---|
| 0.1 | 25 × 25 | 0.31 |
| 0.1 | 50 × 50 | 0.24 |
| 0.1 | 75 × 75 | 0.19 |
| 0.15 | 25 × 25 | 0.28 |
| 0.15 | 50 × 50 | 0.20 |
| 0.15 | 75 × 75 | 0.14 |

### 8.3.7 A Multiplexed Bioassay Example

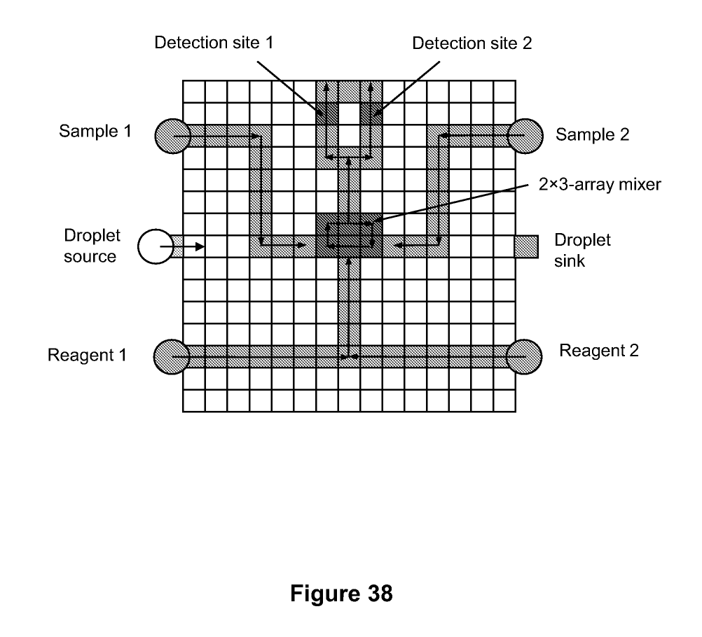

A real-life application was also considered by the inventors, namely a multiplexed biochemical assay consisting of a glucose assay and a lactate assay based on colorimetric enzymatic reactions, which have been demonstrated recently [15]. Referring back to FIG. 18, FIG. 18 shows the flowchart for the multiplexed assays in the form of a sequencing graph [34]. For each sample or reagent, two droplets are dispensed into the array. Four pairs of droplets, i.e., {S₁, R₁}, {S₁, R₂}, {S₂, R₁}, {S₂, R₂} are routed together in sequence for the mixing operation. Mixed droplets are finally routed to detection site for analysis. In [7], the multiplex bioassays were mapped to a digital microfluidic platform containing a 15×15 array, as shown in FIG. 38. A depiction of the droplet pathways for multiplexed glucose and lactase assays is given in FIG. 38.

For simplicity, the mixing and detection operations are ignored and the dispensing of droplets and their transportation to the mixer is focused upon. These steps are referred to as the droplet transportation steps of the bioassay. As a baseline, droplets are first transported by moving only one droplet at a time. It is understood that two droplets must typically be dispensed and routed to the mixer for each sample or reagent, therefore the total time required is simply the sum of times needed to transport each droplet. A total of 8 droplets must be transported at the rate of 0.33 s/droplet, hence the total transportation time is 35 seconds. Next, the array is assumed to be controlled using a direct-addressing scheme with 225 control pins. In this case, droplets can be moved concurrently on the array and the dispensing and routing operation takes only 7 seconds.

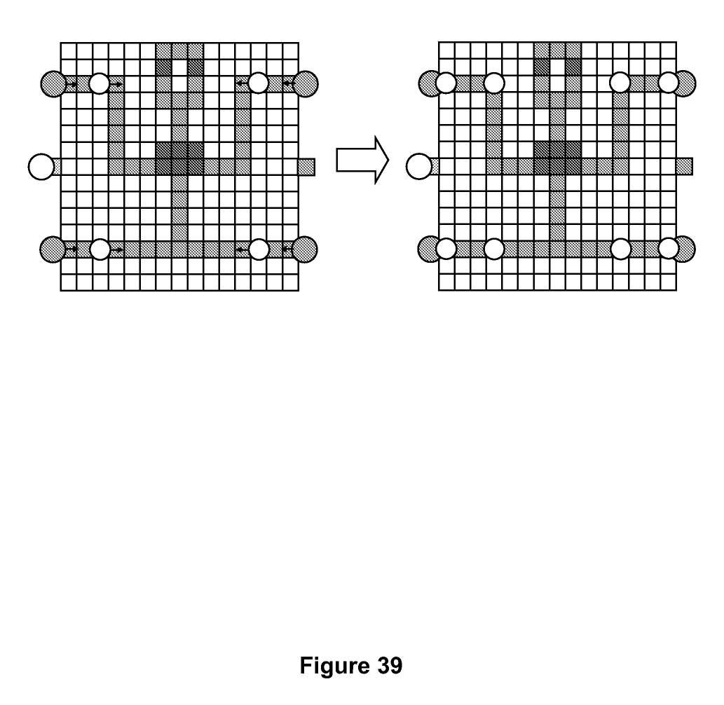

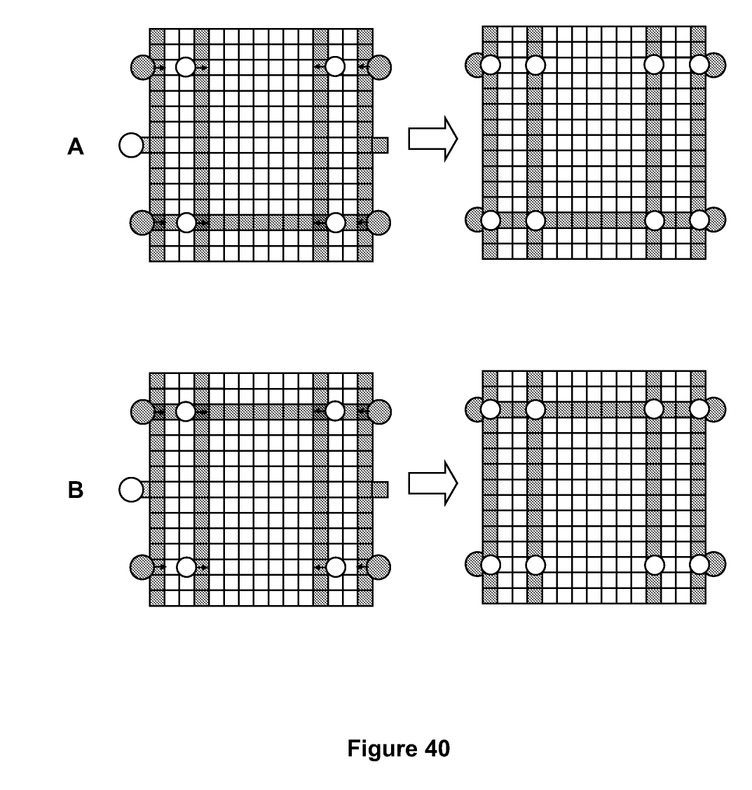

Finally, the droplet operations method that is disclosed herein is applied based on clique partitioning to the example of multiplexed bioassays. The droplet positions for the different time-steps considered herein correspond to the succession of droplet positions obtained using the direct-addressing method. Note that the transition between two time-steps, which takes only one droplet operations step for direct addressing, can sometimes be carried out in one time-step for the cross-referencing-based method disclosed herein as well. No additional droplet operations steps are typically needed in such cases. For other cases, the method disclosed herein decomposes a single droplet movement step, which is adequate for direct addressing, into a succession of steps determined using destination-cell-based categorization. An example is shown in FIG. 39 and FIGS. 40A and 40B.

In the droplet operations step in FIG. 39, 8 droplet movements, i.e., 4 dispensing and 4 droplet transportation operations, are to be executed simultaneously. When the cross-referencing based method disclosed herein is applied, the 8 movements are categorized into two groups and implemented with two droplet operations steps, as shown in FIG. 40A and FIG. 40B, respectively.

In this manner, the droplet operations method that is disclosed herein is applied to every time-step derived from the direct-addressing scheme, and results in a completion time of 15 seconds. A significant reduction in the assay completion time is therefore obtained compared to the one-at-a-time baseline method. This improvement is even more significant if the fact is considered that for the one-at-a-time droplet operations method, droplet routing can be carried out while mixing is being carried at some place on the array.

Moreover, if multiple copies of the same modules, e.g., the one shown in FIG. 18, are placed in parallel on the array, which is a very common “regularization” strategy in VLSI design, the droplet movement time using the method disclosed herein is not affected. In contrast, the one-at-a-time droplet operations method results in an n-fold increase in the assay completion time if n copies of the module in FIG. 18 are mapped to the array. Note that the completion time obtained using the droplet operations method that is disclosed herein is slightly more than that for direct-addressing method (15 seconds versus 7 seconds). However, the method disclosed herein typically requires only 30 (15+15) control pins while 225 (15×15) pins are required for the direct-addressing method.

8.4 Operation Fluids

For examples of fluids that may be subjected to droplet operations using the approach of the invention, see the patents listed in section 2, especially International Patent Application No. PCT/US2006/047486, entitled, “Droplet-Based Biochemistry,” filed on Dec. 11, 2006. In some embodiments, the fluid includes a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, fluidized tissues, fluidized organisms, biological swabs, biological washes, liquids with cells, tissues, multicellular organisms, single cellular organisms, protozoa, bacteria, fungal cells, viral particles, organelles. In some embodiment, the fluid includes a reagent, such as water, deionized water, saline solutions, acidic solutions, basic solutions, detergent solutions and/or buffers. In some embodiments, the fluid includes a reagent, such as a reagent for a biochemical protocol, such as a nucleic acid amplification protocol, an affinity-based assay protocol, a sequencing protocol, and/or a protocol for analyses of biological fluids.

The fluids may include one or more magnetically responsive and/or non-magnetically responsive beads. Examples of droplet actuator techniques for immobilizing magnetically responsive beads and/or non-magnetically responsive beads are described in the foregoing international patent applications and in Sista, et al., U.S. Patent Application No. 60/900,653, entitled “Immobilization of Magnetically-responsive Beads During Droplet Operations,” filed on Feb. 9, 2007; Sista et al., U.S. Patent Application No. 60/969,736, entitled “Droplet Actuator Assay Improvements,” filed on Sep. 4, 2007; and Allen et al., U.S. Patent Application No. 60/957,717, entitled “Bead Washing Using Physical Barriers,” filed on Aug. 24, 2007, the entire disclosures of which is incorporated herein by reference.

Concluding Remarks

The foregoing detailed description of embodiments refers to the accompanying drawings, which illustrate specific embodiments of the invention. Other embodiments having different structures and operations do not depart from the scope of the invention. This specification is divided into sections for the convenience of the reader only. Headings should not be construed as limiting of the scope of the invention. The definitions are intended as a part of the description of the invention. It will be understood that various details of the invention may be changed without departing from the scope of the invention. Furthermore, the foregoing description is for the purpose of illustration only, and not for the purpose of limitation, as the invention is defined by the claims as set forth hereinafter.

### REFERENCES

[1] V. Srinivasan et al., “An integrated digital microfluidic lab-on-a-droplet actuator for clinical diagnostics on human physiological fluids” Lab on a Droplet actuator, pp. 310-315, 2004.
[2] S. K. Fan et al., “Manipulation of multiple droplets on N×M grid by cross-reference EWOD driving scheme and pressure-contact packaging”, Proc. IEEE MEMS Conf., pp. 694-697, 2003.
[3] W. Hwang et al., “Automated design of pin-constrained digital microfluidic arrays for lab-on-a-droplet actuator applications,” Proc. DAC, pp. 925-930, 2006.
[4] R. B. Fair et al., “Electrowetting-based on-droplet actuator sample processing for integrated microfluidics”, Proc. IEDM, pp. 32.5.1-32.5.4, 2003.
[5] E. Verpoorte and N. F. De Rooij, “Microfluidics meets MEMS”, Proc. IEEE, vol. 91, pp. 930-953, 2003.
[6] R. B. Fair et al., “Chemical and biological applications of digital-microfluidic devices”. Proc. IEEE Design & Test of Computers, vol. 24, pp. 10-24, 2007.
[7] T. H. Schulte et al., “Microfluidic technologies in clinical diagnostics”, Clinica Chimica Acta, vol. 321, pp. 1-10, 2002.
[8] R. B. Fair et al., “Integrated chemical/biochemical sample collection, pre-concentration, and analysis on a digital microfluidic lab-on-a-droplet actuator platform”, Lab-on-a-Droplet actuator: Platforms, Devices, and Applications, Conf. 5591, SPIE Optics East, 2004.
[9] Y. Zhao and S. K. Cho, “Microparticle sampling by electrowetting-actuated droplet sweeping”, Lab on a Droplet actuator, vol. 6, pp. 137-144, 2006.
[10] R. B. Fair et al., “Chemical and biological applications of digital microfluidic devices”, accepted for publication in IEEE Design & Test of Computers, 2006.
[11] Advanced Liquid Logic, Inc., http://www.liquid-logic.com
[12] L. Benini, C. Guiducci and C. Paulus, “Electronic detection of DNA hybridization: toward CMOS microarrays”, Proc. IEEE Design & Test of Computers, vol. 24, pp. 38-48, 2007.
[13] Y. Wang et al., “Composable Behavioral Models and Schematic-Based Simulation of Electrokinetic Lab-on-a-Droplet actuator Systems”, IEEE TCAD, vol. 25, pp. 258-273, February 2006.
[14] P. Y. Paik et al., “Rapid droplet mixers for digital microfluidic systems”, Lab on a Droplet actuator, vol. 3, pp. 253-259, 2003.
[15] J. Zeng and T. Korsmeyer, “Principles of droplet electrohydrodynamics for lab-on-a-droplet actuator”, Lab on a Droplet actuator, pp. 265-277, 2004.
[16] K. Chakrabarty and F. Su, “Digital Microfluidic Droplet actuators: Synthesis, Testing, and Reconfiguration Techniques”, CRC Press, FL, 2006.
[17] V. Srinivasan et al., “Clinical diagnostics on human whole blood, plasma, serum, urine, saliva, sweat, and tears on a digital microfluidic platform”, Proc. μTAS, pp. 1287-1290, 2003.
[18] Silicon Biosystems, http://www.siliconbiosystems.com
[19] Advanced Liquid Logic, Inc., http://www.liquid-logic.com
[20] T. Xu and K. Chakrabarty, “Droplet-trace-based array partitioning and a pin assignment algorithm for the automated design of digital microfluidic droplet actuators”, IEEE/ACM CODES+ISSS, pp. 112-117, 2006.
[21] K. F. Böhringer, “Modeling and controlling parallel tasks in droplet-based microfluidic systems”, IEEE TCAD, vol. 25, pp. 329-339, 2006.
[22] E. J. Griffith et al., “Performance characterization of a reconfigurable planar array digital microfluidic system”, IEEE TCAD, vol. 25, pp. 340-352, 2006.
[23] A. J. Ricketts et al., “Priority scheduling in digital microfluidics-based droplet actuators”, Proc. DATE, vol. 1, pp. 1-6, 2006.
[24] P.-H. Yuh et al., “Placement of digital microfluidic droplet actuators using the T-tree formulation”, Proc. DAC, 2006, pp. 931-934.
[25]H. Moon et al., “Integrated digital microfluidic droplet actuator for multiplexed proteomic sample preparation and analysis by MALDI-MS”, Lab on a Droplet actuator, vol. 6, pp. 1213-1219, 2006.
[26] T. Xu and K. Chakrabarty, “A cross-referencing-based droplet manipulation method for high-throughput and pin-constrained digital microfluidic arrays”, Proc. DAte, pp. 552-557, 2007.
[27] P. Y. Paik et al., “Coplanar digital microfluidics using standard printed circuit board processes”, Proc. μTAS, pp. 566-568, 2005.
[28] F. Su, W. Hwang and K. Chakrabarty, “Droplet routing in the synthesis of digital microfluidic droplet actuators” Proc. DATE Conf, pp. 323-328, 2006.
[29] Connect5 strategies, www.springfrog.com/games/gomoku/
[30] R. Diestel, Graph Theory, Springer, Berlin, 2005.
[31] J. L. Gross and J. Yellen, Graph Theory and Its Applications, CRC Press, FL, 1999.
[32] M. R. Garey and D. S. Johnson, Computers and Intractability: A Guide to the Theory of NP-Completeness, W. H. Freeman & Co., CA, 1979.
[33] F. Su et al., “Droplet routing in the synthesis of digital microfluidic droplet actuators”, Proc. DATE Conference, pp. 323-328, 2006.
[34] F. Su and K. Chakrabarty, “Architectural-level synthesis of digital microfluidics-based droplet actuators”, Proc. IEEE ICCAD, pp. 223-228, 2004.

1-88. (canceled)

89. A method of defining partitions for pin layouts in a droplet actuator for a specific assay, the method comprising:
(a) defining droplet traces for the assay; and
(b) defining a guard ring along the traces.

90. The method of claim 89 further comprising:
(a) identifying droplet time spans for each partition; and
(b) merging partitions that have no overlapping time spans.

91. The method of claim 89 further comprising:
(a) identifying intersecting droplet traces;
(b) creating an overlapping partition comprising intersecting electrodes; and
(c) directly addressing the intersecting electrodes.

92. The method of claim 89 further comprising:
(a) identifying mixer regions and/or splitter regions; and
(b) assigning overlapping partitions to the mixer regions and/or splitter regions.

93. The method of claim 89 further comprising mapping pins to control electrodes based on the Connect-5 algorithm.

94. The method of claim 89 further comprising mapping pins to control electrodes comprising assigning distinct pins to any five adjacent unit cells.

95. The method of claim 89 further comprising mapping pins to control electrodes comprising placing Bagua structures next to each other until partition boundaries are reached and a Bagua repetition is derived.

96. A droplet actuator configured according to the method of claim 89.

97. A method of conducting an assay comprising conducting the assay on a droplet actuator configured according to the method of claim 89.

98. A method of conducting an assay comprising conducting the assay on a droplet actuator configured according to the method of claim 89 wherein the time required to conduct the assay is approximately the same as the time required to conduct the assay using independently addressed electrodes.

99. A method for performing one or more droplet operations using an electrical field generated by a droplet actuator having a plurality of control pins used to electrically actuate a plurality of electrodes configured to communicate the electrical field to the droplet, the method comprising:
(a) receiving a plurality of respective activation sequences for each electrode of the plurality electrodes;
(b) identifying a compatible activation sequence that is compatible with the plurality of respective activation sequences by logically manipulating a signal value used to determine a status of an electrode at a given time-step of the respective activation sequence of the electrode;
(c) communicating the compatible activation sequence to the plurality of electrodes using a common control pin of the plurality of control pins; and
(d) performing one or more droplet operations according to the compatible activation sequence.

100. A droplet actuator for performing one or more droplet operations using an electrical field, the method comprising:
(a) a plurality of electrodes configured to communicate the electrical field to the droplet;
(b) a plurality of control pins used to electrically actuate the plurality of electrodes; and
(c) a processor in communication with both the plurality of electrodes and control pins and configured to receive a plurality of respective activation sequences for each electrode of a plurality electrodes, to automatically identify based on the received activation sequences a compatible activation sequence that is compatible with the plurality of respective activation sequences, to communicate the compatible activation sequence to the plurality of electrodes using a common control pin of a plurality of control pins, and to perform droplet operations according to the compatible activation sequence.

101-114. (canceled)

115. A method of performing one or more droplet operations using an electrical field generated by a droplet actuator having rows and columns corresponding to electrodes configured to communicate the electrical field to the droplet, the method comprising:
(a) logically grouping a plurality of droplets into first and second groups; and
(b) concurrently moving a plurality of droplets of the first group, while a droplet of the second group is stationary.

116-131. (canceled)

132. A droplet actuator for performing one or more droplet operations using an electrical field, comprising:
(a) a plurality of electrodes arranged in rows and columns and configured to communicate the electrical field to the droplet; and
(b) a processor in communication with the plurality of electrodes and configured to logically group a plurality of droplets into first and second groups, and to concurrently initiate movement of a plurality of droplets in the first group, while a droplet of a second group is stationary.

133-148. (canceled)
